(19) United States
(12) Patent Application Publication
(10) Pub. No.: US 2009/0317407 A1
(43) Pub. Date: Dec. 24, 2009

(54) AUGMENTATION OF IMMUNE RESPONSE TO CANCER VACCINE

(76) Inventors: Michael G. LaCelle, Portland, OR (US); Bernard A. Fox, Portland, OR (US); Shawn M. Jensen, Carnas, WA (US); Christian H. Poehlein, Portland, OR (US)

Correspondence Address: KLARQUIST SPARKMAN, LLP, 121 SW SALMON STREET, SUITE 1600, PORTLAND, OR, 97204, US

(21) Appl. No.: 12/296,521

(22) PCT Filed: May 2, 2007
(86) PCT No.: PCT/US07/10798

§ 371 Date: Oct. 8, 2008

Related U.S. Application Data
(60) Provisional application No. 60/797,361, filed on May 2, 2006.

(51) Int. Cl. A61K 39/395; A61K 39/00; A61P 35/00

(52) U.S. Cl. 424/174.1; 424/184.1

(57) ABSTRACT

Methods are disclosed for stimulating an immune response against a target antigen, such as one or more tumor associated antigens. In particular examples the method includes reducing the number of CD4+ T cells in a subject after the subject receives a first dose of an immunogenic composition that includes the target antigen (such as a cancer vaccine), wherein the subject has a tumor that expresses the target antigen. The subject is administered a second dose of the immunogenic composition, thereby stimulating an immune response against the target antigen. In some examples the method also includes administering to the subject peripheral blood mononuclear cells (PBMCs) (for example a population of PBMCs depleted of CD25 cells, CD81 cells, CD134⁺ cells, Areg⁺ cells, Ptgr3⁺ cells, or combinations thereof) prior to receiving the first dose of the immunogenic composition.

### STATEMENT OF GOVERNMENT SUPPORT

This invention was made with United States government support pursuant to grants NIH RO1-CA 80964. The United States government has certain rights in the invention.

### CROSS-REFERENCE TO RELATED APPLICATIONS

This application claims the benefit of US Provisional Application No. filed May 2, 2006, herein incorporated by reference.

### FIELD

This application relates to methods of augmenting an immune response to an immunogenic composition that includes one or more tumor antigens (such as a cancer vaccine), for example by reducing the number of CD4 cells in a subject following the first administration of the immunogenic composition.

### BACKGROUND

Over 1.3 million new cancer patients will be diagnosed in 2005 and 570,280 will die of the disease. Classic treatments have been refined and overall survival rates have improved, but better treatments are needed. T cell-based immunotherapy strategies (such as cancer vaccines) have been effective therapeutic modality in animal models. However, their translation into the clinic, with some exceptions, has been disappointing.

Therefore, therapies that enhance immunotherapy strategies to treat cancer are needed.

### SUMMARY

Provided herein are methods that can be used to stimulate, such as enhance or augment, an immune response against a target antigen, such as one or more tumor antigens or pathogen antigens. In particular examples, the method includes reducing or depleting CD4+ T cells in a subject, at a time subsequent to the subject receiving a first dose of a therapeutically effective amount of an immunogenic composition that includes the target antigen. A therapeutically effective amount of a second dose of the immunogenic composition is administered to the subject, thereby stimulating an immune response against the target antigen. The subject can have a tumor that expresses one or more of the target antigens, or may have had the tumor removed (for example by surgery or chemotherapy). Therefore, the immunogenic composition is selected based on the tumor in the subject. For example, if the subject has a prostate cancer (or has had a prostate tumor removed), the immunogenic composition includes one or more prostate-specific antigens. In a particular example, the immunogenic composition is a cancer vaccine.

Exemplary tumors that can be targeted include benign and cancerous tumors, such as breast cancer, melanoma, lung cancer, renal cell carcinoma, prostate cancer, ovarian cancer, cervical cancer, colon cancer, a liver cancer, or combinations thereof.

In particular examples, reducing or depleting the number of CD4+ T cells in the subject is performed in vivo, for example by administering to the subject a therapeutically effective amount of an agent that significantly reduces the number of CD4+ T cells in the subject under conditions sufficient to reduce the number of CD4+ T cells in the subject. However, 100% depletion is not required. For example depletion of at least 30%, at least 50%, or at least 70% of the CD4+ T cells can be sufficient. Exemplary agents that can be used to deplete CD4 cells include agents that significantly decrease the biological activity of CD4, such as CD4 antibodies, CD4 immunotoxins, antisense, and siRNA molecules. However, one skilled in the art will appreciate that such methods can also be performed ex vivo.

Depletion of the CD4+ T cells and the administration of the second dose of the immunogenic composition can occur simultaneously. In other examples, the depletion of CD4+ T cells occurs prior to administration of the second dose, such as at least 6 hours, at least 24 hours, or at least 48 hours prior to administration of the second dose of the immunogenic composition.

In particular examples, the method also includes reconstituting the subject with peripheral blood mononuclear cells (PBMCs), such as autologus PBMCs obtained from the subject previously. In one example, the subject is administered peripheral PBMCs prior to or at essentially the same time when the subject received a first dose of the immunogenic composition. In some examples, the subject is also lymphodepleted prior to administering the first dose of the immunogenic composition and the PBMCs, and following apheresis of the subject used to obtain the PBMCs.

The PBMCs can be depleted of regulatory T cells (T_{reg}) and/or tumor-induced regulatory T cells (iT_{reg}), such as CD25+ cells, CD81+ cells, CD134+ cells, amphiregulin (Areg)+ cells, prostaglandin receptor EP3 (Ptger3)+ cells, or combinations thereof. Such depletion can be performed ex vivo, for example by contacting PBMCs obtained from the subject with antibodies specific for CD25, CD81, CD134, Areg, Ptger3, or combinations thereof (such as two or more markers), thereby removing the CD25+, CD81+, CD134+, Areg+, or Ptger3+ cells. However, such depletion does not require 100% depletion. In some examples, depletion of at least 30% is sufficient, such as at least 50%, at least 75%, at least 95%, or at least 99% depletion.

Also provided by the present disclosure are kits that include one or more agents for depleting CD4+ T cells, such as an anti-CD4 antibody. The kit can further include one or more agents for depleting iTreg cells, such a CD25+, CD81+, CD134+, Areg+, Ptger3+ cells, or combinations thereof, for example an anti-CD25, anti-CD81, anti-CD134, anti-Areg, or anti-Ptger3 antibody (or combinations thereof). In some examples, the kit also includes an immunogenic composition, such as a cancer vaccine.

### BRIEF DESCRIPTION OF THE DRAWINGS

FIG. 1 is a schematic drawing showing the experimental design used to obtain the results shown in FIG. 2.

FIG. 2 is a plot showing that administration of anti-CD4 mAb at second and third vaccines augments therapeutic efficacy of the vaccine.

FIG. 3 shows tumor growth in wild-type (A, C) or lymphopenic mice deficient of T_{reg} (Rag1−/−) (B, D) administered vaccine alone (A, B) or in combination with T cells (C and D). Each line represents the size of one tumor in an individual mouse.

FIG. 4 is a schematic drawing of a clinical trial protocol for men with advanced hormone-refractory prostate cancer (HRPC) using both a prostate cancer vaccine and anti-CD4 monoclonal antibodies.

FIG. 5A is a schematic showing how defective ribosomal products in blebs (DRibbles) released from cells (such as tumor cells) after proteasome inhibitor-induced autophagy (or starvation) can accumulate defective ribosomal products (DRiPs) and short lived proteins (SLiPs) (and fragments thereof) in autophagy bodies.

FIG. 5B is a series of graphs showing that treatment of mice having breast cancer tumors is enhanced with both a DRibble vaccine and anti-CD4 are used.

FIG. 6 is a schematic showing the experimental design used to demonstrate that vaccination of reconstituted lymphopenic mice (RLM) reconstituted with spleen cells from tumor bearing mice (TBM) is not effective.

FIG. 7 is a showing the experimental design used to obtain the results shown in FIGS. 8-10.

FIG. 8 is a bar graph showing that depletion of CD25 cells from TBM RLM restores tumor-specific cytokine (INF-γ) release from effector T cells (TE) generated in RLM. Data shown represents the means (SEM) of 2 consecutive experiments.

FIG. 9 is a digital image of lungs from mice having tumors and treated as shown. This figure demonstrates that depletion of CD25+ cells from TBM spleen used to reconstitute lymphopenic hosts restores therapeutic efficacy in adoptive immunotherapy (AIT).

FIG. 10 is a bar graph showing that the tumor-specific cytokine response of TE is restored when TE are generated in RLM reconstituted with CD25-depleted TBM spleen cells even when used to reconstitute lymphopenic TBM with progressive tumor burden as RLM hosts. Data shows the means (SEM) of 2 consecutive experiments of 24 hour tumor-specific IFN-γ release measured by ELISA.

FIG. 11 shows flow cytometric analysis of TBM CD3+ CD4+CD25+(gated through G1&G2&G3&G4) and TBM CD3+ CD4+CD25− (gated through G1&G2&G3&G5) spleen-derived T cells for surface expression of markers identified in gene microarray analyses.

FIG. 12 is a series of bar graphs comparing the surface expression of several proteins on TBM and naïve CD3+ CD4+CD25+ and CD3+ CD4+CD25− spleen-derived T cells. Data is presented as % of all CD4+ T cells in 3 consecutive paired experiments.

FIG. 13 is a bar graph showing that magnetic bead depletion of CD25+, CD81+, CD134+ and GITR+ TBM spleen cells prior to reconstitution are all equally effective in restoring the generation of tumor-specific TE in the RLM. In contrast, depletion of CD137+CD152+, CD38+ or LAG-3+ subsets delivers only miner or no recovery. Data shows 24 h tumor-stimulated IFN-γ secretion evaluated by ELISA.

FIG. 14 is a bar graph showing that inhibition of the generation of D5 tumor-specific TE in the RLM is induced by iTreg from multiple different, syngenic but unrelated tumors. Data represents the mean (SEM) of two consecutive experiments.

FIGS. 15-17 are schematic drawings representing clinical trials for (15) Cohort A having CD25, (16) Cohort B receiving autologous, unmanipulated PBMC and systemic doses of zanolimumab, and (17) Cohort C having both ex vivo CD25 depletion of PBMC from a pheresis pack and in vivo CD4 depletion with systemic zanolimumab.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a tumor antigen” includes single or plural tumor antigen and is considered equivalent to the phrase “comprising at least one tumor antigen.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs.

CD4: cluster of differentiation factor 4

CD25: cluster of differentiation factor 25

CD81: cluster of differentiation factor 81

CD134: cluster of differentiation factor 134

DC: dendritic cells

HRPC: hormone-refractory prostate cancer

iTreg: tumor-induced regulatory T cells

PBMC: peripheral blood mononuclear cell

ptger3: prostaglandin receptor EP3

RLM: reconstituted lymphopenic mice

TE: effector T cells

Treg: regulatory T cells

TVDLN: tumor vaccine-draining lymph nodes

Adjuvant: An agent that when used in combination with an immunogenic agent (such as a vaccine, for example a cancer vaccine) augments or otherwise alters or modifies a resultant immune response. In some examples, an adjuvant increases the titer of antibodies induced in a subject by the immunogenic agent. In another example, if the antigenic agent is a multivalent antigenic agent, an adjuvant alters the particular epitopic sequences that are specifically bound by antibodies induced in a subject.

Exemplary adjuvants that can be used with a vaccine include, but are not limited to, Freund's Incomplete Adjuvant (IFA), Freund's complete adjuvant, B30-MDP, LA-15-PH, monophosphoryl/Lipid A (MPL), Poly I:C, montanide, saponin, aluminum salts such as aluminum hydroxide (Amphogel, Wyeth Laboratories, Madison, N.J.), alum, lipids, keyhole lympet protein, hemocyanin, edestin, the MF59 microemulsion, a mycobacterial antigen, vitamin E, non-ionic block polymers, muramyl dipeptides, polyanions, amphipatic substances, ISCOMs (immune stimulating complexes, such as those disclosed in European Patent EP 109942), vegetable oil, Carbopol, aluminium oxide, oil-emulsions (such as Bayol F or Marcol 52), bacterial toxins (such as B. anthracis protective antigen, E. coli heat-labile toxin (LT), Cholera toxin, tetanus toxin/toxoid, diphtheria toxin/toxoid, P. aeruginosa exotoxin/toxoid/, pertussis toxin/toxoid, and C. perfringens exotoxin/toxoid), bacterial wall proteins and other products (such as cell walls and lipopolysaccharide (LPS)) and combinations thereof.

In one example, the adjuvant includes a DNA motif that stimulates immune activation, for example the innate immune response or the adaptive immune response by T-cells, B-cells, monocytes, dendritic cells, and natural killer cells. Specific, non-limiting examples of a DNA motif that stimulates immune activation include CpG oligodeoxynucleotides, as described in U.S. Pat. Nos. 6,194,388; 6,207,646; 6,214,806; 6,218,371; 6,239,116; 6,339,068; 6,406,705; and 6,429,199, and GM-CSFor other immunomodulatory cytokines, such as IL-2, IL-7, IL-15 and IL-21.

In one example, the adjuvant includes ssRNA or dsRNA, such as ssRNA single strand oligoribonucleotides (ORN). For example, an adjuvant can include a GU-rich RNA from HIV (such as GCCCGUCUGUUGUGUGACUC; SEQ ID NO: 1; Science 303(5663):1526-9, 2004).

In another example, a synthetic adjuvant includes R848 (a TLR7/8 ligand) (3M pharmaceutical) or α-galcer (a NKT cell ligand).

Administration: To provide or give a subject an agent, such as an immunogenic composition (such as a cancer vaccine), an agent that depletes CD4+ T cells, or an agent that depletes iT_{reg} cells, by any effective route. Exemplary routes of administration include, but are not limited to, oral, injection (such as subcutaneous, intramuscular, intradermal, intraperitoneal, and intravenous), sublingual, rectal, transdermal, intranasal, vaginal and inhalation routes.

Amphiregulin (Areg): A homolog member of the EGF-like family and ligand for the epithelial growth factor receptor (EGFR). Areg can stimulate through the EGFR and promote and cell survival and suppress apoptosis similar to EGF/EGFR interactions with epithelial and tumor cells. Areg has been shown to inhibit apoptosis induction in epithelial cells and non-small cell lung cancer cells. Sequences for Areg are publicly available (for example, exemplary Areg mRNA sequences are available from GenBank Accession Nos: NM_{—}009704.3 and NM_{—}001657.2, and exemplary Areg protein sequences are available from GenBank Accession Nos: NP_{—}033834.1, EAX05710.1, and NP_{—}001648.1). Antibodies specific for Areg are publicly available (for example from Abcam, Cambridge, Mass. and R&D MAB262). In particular examples, antibodies specific for Areg or other agents that reduce or inhibit Areg activity are used to deplete PBMCs of iT_{reg} or administered in vivo to deplete or to reduce the generation or activity of iT_{reg}.

Antibody: A molecule including an antigen binding site which specifically binds (immunoreacts with) an antigen. Includes immunoglobulin molecules and immunologically active portions thereof. Immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Antigen: A substance that can stimulate the production of antibodies or a T-cell response in a mammal, including compositions that are injected or absorbed into a mammal. An antigen reacts with the products of specific humoral or cellular immunity, including those induced by heterologous immunogens. The term “antigen” includes all related antigenic epitopes. In one example, an antigen is a cancer antigen. A target antigen is an antigen against which an immune response is desired, for example to achieve a therapeutic effect, such as tumor regression.

Antigen-specific T cell: A CD8⁺ or CD4⁺ lymphocyte that recognizes a particular antigen. Generally, antigen-specific T cells specifically bind to a particular antigen presented by MHC molecules, but not other antigens presented by the same MHC.

Cancer: Malignant neoplasm that has undergone characteristic anaplasia with loss of differentiation, increased rate of growth, invasion of surrounding tissue, and is capable of metastasis.

CD4 (cluster of differentiation factor 4): A T-cell surface protein that mediates interaction with MHC class II molecules. This cell surface antigen is also known as T4, Leu-3, OKT4 or L3T4. CD4 is a 55 kDa transmembrane glycoprotein belonging to the immunoglobulin superfamily. A T-cell that expresses CD4 is a “CD4+” T-cell. Likewise, a T-cell that does not express CD4 is a “CD4⁻” T-cell. Sequences for CD4 are publicly available (for example, exemplary CD4 mRNA sequences are available from GenBank Accession Nos: NM_{—}013488.2, NM_{—}000616.3, and NM_{—}001009250.1, and exemplary CD4 protein sequences are available from GenBank Accession Nos: NP_{—}038516.1, NP_{—}036837.1, and NP_{—}000607.1). Antibodies specific for CD4 are publicly available (for example HuMax-CD4 (zanolimumab) from Serono S A and Genmab, Denmark). Other CD4 inhibitory molecules, such as siRNAs are known (for example see Novina et al., Nature Med. 8: 681-6, 2002, and from Sigma, St. Louis, Mo.). In particular examples, antibodies specific for CD4 or other agents that reduce or inhibit CD4 activity are used to deplete CD4 cells in vivo.

CD25 (cluster of differentiation factor 25): The IL-2 receptor alpha chain (IL-2 receptor alpha subunit, IL-2-RA), which is expressed on T regulatory cells. CD25 was the first marker identified that can distinguish CD4⁺ regulatory T cells from Th1 helper T cells. A T-cell that expresses CD25 is a “CD25+” T cell. Sequences for CD25 are publicly available (for example, exemplary CD25 mRNA sequences are available from GenBank Accession Nos: NM_{—}001009355.1 and NM_{—}000417, and exemplary CD25 protein sequences are available from GenBank Accession Nos: P01589, NP_{—}000408, NP_{—}001009355.1, and P01590). Antibodies specific for CD25 are publicly available (for example from RDI Division of Fitzgerald Industries Intl., Concord, Mass.). In particular examples, antibodies specific for CD25 or other agents that reduce or inhibit CD25 activity are used to deplete PBMCs of iT_{reg}.

CD81 (cluster of differentiation factor 81): A 26 kDa non-glycosylated member cell-surface protein of the tetraspanin superfamily, which is a coreceptor in B and T cell activation. Also known as target of the antiproliferative antibody 1 (TAPA1). CD81 can enhance Th1 and Th2 stimulation, and preferentially support Th2 signaling found in the central zone of the T cell/APC immunological synapse. A T-cell that expresses CD81 is a “CD81+” T cell. Sequences for CD81 are publicly available (for example, exemplary CD81 mRNA sequences are available from GenBank Accession Nos: BC093047, NM_{—}013081.1, NM_{—}004356.3, and NM_{—}133655.1, and exemplary CD81 protein sequences are available from GenBank Accession Nos: NP_{—}598416.1, AAH93047, AAH60583.1, and NP_{—}004347.1). Antibodies specific for CD81 are publicly available (for example from Novocastra, United Kingdom). In particular examples, antibodies specific for CD81 or other agents that reduce or inhibit CD81 activity are used to deplete PBMCs of iT_{reg}.

CD134 (OX40R) (cluster of differentiation factor 134): A T-cell glycoprotein antigen structurally belonging to the tumor necrosis factor receptor gene family. CD134 is a secondary costimulatory molecule, expressed after 24 to 72 hours following activation. A T-cell that expresses CD134 is a “CD134+” T cell. Sequences for CD134 are publicly available (for example, exemplary CD134 mRNA sequences are available from GenBank Accession Nos: AJ277151.1 and AY738589.1, and exemplary CD134 protein sequences are available from GenBank Accession Nos: CAB96543.1 and AAU84987.1). Antibodies specific for CD134 are publicly available (for example from RDI Division of Fitzgerald Industries Intl., Concord Mass.). In particular examples, antibodies specific for CD134 or other agents that reduce or inhibit CD134 activity are used to deplete PBMCs of iT_{reg}.

Chemotherapy: In cancer treatment, chemotherapy refers to the administration of one or more agents to kill or slow the reproduction of rapidly multiplying cells, such as tumor or cancer cells. In a particular example, chemotherapy refers to the administration of one or more anti-neoplastic agents to significantly reduce the number of tumor cells in the subject, such as by at least 50%. Cytotoxic anti-tumor chemotherapeutic agents include, but are not limited to: 5-fluorouracil (5-FU), azathioprine, cyclophosphamide (such as Cytoxan®), antimetabolites (such as Fludarabine), and other antineoplastics such as Etoposide, Doxorubicin, methotrexate, Vincristine, carboplatin, cis-platinum and the taxanes (such as taxol).

Decrease or deplete: To reduce the quality, amount, or strength of something.

In one example, a therapy (such as the methods provided herein) decreases a tumor (such as the size of a tumor, the number of tumors, the metastasis of a tumor, or combinations thereof), or one or more symptoms associated with a tumor, for example as compared to the response in the absence of the therapy. In a particular example, a therapy decreases the size of a tumor, the number of tumors, the metastasis of a tumor, or combinations thereof, subsequent to the therapy, such as a decrease of at least 10%, at least 20%, at least 50%, or even at least 90%. Such decreases can be measured using the methods disclosed herein. In a particular example, the disclosed methods can be used to decrease a tumor to a greater extent than administration of a cancer vaccine alone or than lymphodepletion in combination with a cancer vaccine.

In another example, a therapy depletes a population of cells. For example, lymphodepletion involves methods that reduce the number of lymphocytes in a subject, for example by administration of a lymphodepletion agent. Similarly, therapies are provided for depleting or reducing the number of CD4+ T cells in a subject, for example by administration of a CD4 antibody.

Methods are provided for depleting one or more sub-populations from a blood sample, for example depleting a PBMC sample of T_{regs} or iT_{regs}, for example by depleting CD25+, CD81+, CD134+, Areg+, Ptger3+ or combinations thereof (such as two or more of these, such as 2, 3, 4 or 5 of these), for example by incubating the PBMCs with antibodies specific for CD25, CD81, CD134, Areg, or Ptger3, respectively. One skilled in the art will recognize that depletion of sub-populations of cells does not require 100% elimination of the undesired cells. For example, a reduction of at least 20%, at least 40%, at least 50%, at least 90%, at least 95%, or at least 99% can be sufficient.

Enhance: To improve the quality, amount, or strength of something.

In one example, a therapy enhances the immune system if the immune system is more effective at fighting infection or tumors, as compared to immune function in the absence of the therapy. For example, the disclosed methods can be used to enhance the effect of a vaccine, such as compared to administration of the vaccine alone or as compared to vaccine in combination with lymphodepletion.

In a particular example, a therapy enhances the immune system if the number of lymphocytes increases subsequent to the therapy, such as an increase of at least 10%, at least 20%, at least 50%, or even at least 90%. Such enhancement can be measured using methods known in the art for example determining the number of lymphocytes before and after the therapy using flow cytometry.

In yet another example, a therapy enhances the frequency of tumor-specific T cells in a subject, such as an increase of at least 20%, at least 30%, at least 50%, or at least 90%. In a particular example, in the absence of a therapy, the frequency of tumor-specific T cells is undetectable or less than 0.01%, while in the presence of an effective therapy the number of T cells is at least 0.1%, such as at least 10%, wherein the percentage is relative to the total number of T cells in a sample, such as a biological sample obtained from a mammal.

Forkhead Box P3 (Foxp3): A transcription factor that appears to drive CD4⁺ T cells to develop regulatory rather then Th1 helper function. Foxp3 is a discriminator of Treg. Expression of Foxp3 can be determined using routine methods in the art, such as PCR, Western blot and flow cytometry. Sequences for Foxp3 are publicly available (for example, exemplary Foxp3 mRNA sequences are available from GenBank Accession Nos: NM_{—}014009.2, AY376065.1, and NM_{—}054039.1, and exemplary Foxp3 protein sequences are available from GenBank Accession Nos: ABN79272.1, NP_{—}054728.2, and NP_{—}473380.1). Antibodies specific for Foxp3 are publicly available (for example from AbD Serotec, Raleigh, N.C., eBioscience, San Diego, Calif., and United States Biological, Swampscott, Mass.). In particular examples, antibodies specific for Foxp3 or other agents that reduce or inhibit Foxp3 activity are used to deplete PBMCs of iT_{reg} or administered in vivo to deplete or reduce the generation of iT_{reg}.

Harvest: To collect. For example, when harvesting PBMCs, the method can include separating the PBMCs from other blood cells, for example by apheresis.

Immune response: A change in immunity, for example a response of a cell of the immune system, such as a B-cell, T-cell, macrophage, monocyte, or polymorphonucleocyte, to an immunogenic agent in a subject. The response can be specific for a particular antigen (an “antigen-specific response”). In a particular example, an immune response is a T cell response, such as a CD4⁺ response or a CD8⁺ response. In another example, the response is a B-cell response, and results in the production of specific antibodies to the immunogenic agent.

In some examples, such an immune response provides protection for the subject from the immunogenic agent or the source of the immunogenic agent. For example, the response can treat a subject having a tumor, for example by interfering with the metastasis of the tumor or reducing the number or size of a tumor. An immune response can be active and involve stimulation of the subject's immune system, or be a response that results from passively acquired immunity.

In a particular example, an increased or enhanced immune response is an increase in the ability of a subject to fight off a disease, such as a tumor.

Immunity: The state of being able to mount a protective response upon exposure to an immunogenic agent. Protective responses can be antibody-mediated or immune cell-mediated, and can be directed toward a particular pathogen or tumor antigen. Immunity can be acquired actively (such as by exposure to an immunogenic agent, either naturally or in a pharmaceutical composition) or passively (such as by administration of antibodies or in vitro stimulated and expanded T cells).

Immunogen: An agent (such as a compound, composition, or substance) that can stimulate or elicit an immune response by a subject's immune system, such as stimulating the production of antibodies or a T-cell response in a subject. Immunogenic agents include, but are not limited to, tumor associated antigens (TAAs) and pathogen antigens. One specific example of an immunogenic composition is a vaccine (such as a vaccine that includes one or more TAAs).

Immunogenicity: The ability of an immunogen to induce a humoral or cellular immune response. Immunogenicity can be measured, for example, by the ability to bind to an appropriate MHC molecule (such as an MHC Class I or II molecule) and to induce a T-cell response or to induce a B-cell or antibody response, for example, a measurable cytotoxic T-cell response or a serun antibody response to a given epitope. Immunogenicity assays are well-known in the art and are described, for example, in Paul, Fundamental Immunology, 3rd ed., 243-247 (Raven Press, 1993) and references cited therein.

Immunologically Effective Dose: A therapeutically effective amount of an immunogen that will prevent, treat, lessen, or attenuate the severity, extent or duration of a disease or condition, for example, a tumor. In a particular example, an immunologically effective dose includes an amount of a cancer vaccine.

Immunostimulant: An agent that can stimulate an immune response against an antigen. One example is an adjuvant. Other particular examples include a costimulatory antibody of T-cell proliferation and survival, such anti-CTLA-4 (madarex) or anti-OX-40 antibody. For example, a cancer vaccine can be used in combination with an immunostimulant.

Immunosuppression: Nonspecific unresponsiveness of cellular or humoral immunity. Immunosuppression refers to the prevention or diminution of an immune response and occurs when T or B cells are depleted in number or suppressed in their reactivity, expansion or differentiation. Immunosuppression may arise from activation of specific or non-specific Treg cells, from cytokine signaling, in response to irradiation, or by drugs that have generalized immunosuppressive effects on T and B cells.

Interferon-gamma (IFN-γ): A protein produced by T lymphocytes in response to specific antigen or mitogenic stimulation. Includes naturally occurring IFN-γ peptides and nucleic acid molecules and IFN-γ fragments and variants that retain full or partial IFN-γ biological activity. Sequences for IFN-γ are publicly available (for example, exemplary IFN-γ mRNA sequences are available from GenBank Accession Nos: BC070256; AF506749; and J00219, and exemplary IFN-γ protein sequences are available from GenBank Accession Nos: CAA00226; AAA72254; and 0809316A).

Methods of measuring functional IFN-γ are known, and include, but are not limited to: immunoassays. For example, the public availability of antibodies that recognize IFN-γ permits the use of ELISA and flow cytometry to detect cells producing IFN-γ. Another method is a cyotoxicity assay that measures the level of killing of tumor targets by activated T cells (for example see Hu et al., J. Immunother. 27:48-59, 2004, and Walker et al., Clin. Cancer Res. 10:668-80, 2004).

Isolated: An “isolated” biological component (such as a portion of hematological material, for example blood components) has been substantially separated or purified away from other biological components of the organism in which the component naturally occurs.

An isolated cell is one which has been substantially separated or purified away from other biological components of the organism in which the cell naturally occurs. For example, an isolated peripheral blood mononuclear cell (PBMC) is a population of PBMCs which are substantially separated or purified away from other blood cells, such as red blood cells or polynuclear cells.

Lymphodepletion agent: A chemical compound or composition capable of decreasing the number of functional lymphocytes in a mammal when administered to the mammal. One example of such an agent is one or more chemotherapeutic agents. In a particular example, administration of a lymphodepletion agent to a subject decreases T-cells by at least 50%. In particular examples, lymphodepletion agents are administered to a subject prior to administration of an immunogen (such as an immunogenic composition, for example a cancer vaccine), for example to enhance the CTL and HTL expansion and persistence after administration of the immunogen. Lymphodepletion can also be attained by partial body or whole body fractioned radiation therapy.

Lymphotoxin alpha (LT-α): This protein is produced predominantly by mitogen-stimulated T-lymphocytes and leukocytes. LT-α is also secreted by fibroblasts, astrocytes, myeloma cells, endothelial cells, and epithelial cells. The synthesis of LT-α is stimulated by interferons and IL2. Also known as tumor necrosis factor beta (TNF-β). Sequences for LT-α are publicly available (for example, exemplary LT-α mRNA sequences are available from GenBank Accession Nos: NM_{—}000595.2, NM_{—}080769.1, and NM_{—}010735.1, and exemplary LT-α protein sequences are available from GenBank Accession Nos: NP_{—}000586.2, NP_{—}034865.1, and NP_{—}542947.1). Antibodies specific for LT-α are publicly available (for example from eBioscience, San Diego, Calif., and Chemicon, Temecula, Calif.). In particular examples, antibodies or other agents that reduce or inhibit LT-α activity are used to deplete PBMCs of iT_{reg} or administered in vivo to deplete or reduce the generation of iT_{reg}.

Malignant: Cells which have the properties of anaplasia invasion and metastasis.

Neoplasm: Abnormal growth of cells.

Pharmaceutically Acceptable Carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more agents to a subject. The compositions used in the methods disclosed herein, such as immunogenic compositions and compositions that can be used to deplete CD4+ T cells or iT_{regs}, can include one or more pharmaceutically acceptable carriers.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Prostaglandin receptor EP3 (ptger3): A receptor for prostaglandin E2 (PGE2) with a distinct signaling capacity from the more ubiquitously expresses receptors EP2 and EP4. Sequences for ptger3 are publicly available (for example, exemplary ptger3 mRNA sequences are available from GenBank Accession Nos: BC118659.1, NM_{—}011196.2, and NM_{—}001082671.1, and exemplary ptger3 protein sequences are available from GenBank Accession Nos: CAI20228.1, NP_{—}035326.2, and P34980). Antibodies specific for ptger3 are publicly available (for example from Abcam, Cambridge, Mass., and Sigma, St. Louis, Mo.). In particular examples, antibodies or other agents that reduce or inhibit ptger3 activity are used to deplete PBMCs of iT_{reg} or administered in vivo to deplete or reduce the generation of iT_{reg}.

Pulsatile Dose: A dose administered as a bolus. A pulsatile dose can be administered to a subject as a single administration, such as by direct injection or by an intravenous infusion during a specified time period. Thus, the pulsatile dose can be a “push” or rapid dose, but need not be, as it can be administered over a defined time period, such as in an infusion. Repeated pulsatile doses (for example of a vaccine) can be administered to a subject, such as a bolus administered repeatedly, such as about every one, two, or three months, or about every one, two, three or four weeks or about every one, two or three days in a therapeutic regimen. In this example, the administered dose can be the same amount of an agent, or can be different amounts administered at several time points separated by periods wherein the agent is not administered to the subject, or wherein a decreased amount of the agent is administered to the subject.

Regulatory T Cells (Treg): T cells that reduce or prevent the activation or expansion of other cell populations and express Foxp3 (for example see Fontenot et al., Nature Immunol. 4:330-36, 2003; Hori et al., Science 299:1057-61, 2003), for example CD4+ CD25+T cells. Reduction or functional alteration of Treg cells leads to the spontaneous development of various organ-specific autoimmune diseases, including, for example, autoimmune thyroiditis, gastritis, and type 1 diabetes (see, for example, Sakaguchi et al., J. Immunol. 155:1151-64, 1995; Suri-Payer et al., J. Immunol. 160:1212-18, 1998; Itoh et al., J. Immunol. 162:5317-26, 1999). In particular examples, Treg cells expess CD81, amphiregulin (a ligand for EGF receptor), prostaglandin receptor EP3 (ptger3), CD25, and LT-α. Tumor-induced regulatory T cells are referred to as iTreg cells.

Stimulate proliferation: To increase the growth or reproduction of cells, for example to increase the number of antigen-specific T cells.

Subject: Living multi-cellular vertebrate organisms, a category that includes human and non-human mammals (such as laboratory or veterinary subjects, for example cats, dogs, rodents, cows, sheep, and horses).

Therapeutically effective amount: An amount of an agent that alone, or together with a pharmaceutically acceptable carrier or one or more additional therapeutic agents, induces the desired response. A therapeutic agent, such as a vaccine, is administered in therapeutically effective amounts that stimulate a protective immune response, for example against a target antigen.

Effective amounts a therapeutic agent can be determined in many different ways, such as assaying for an increase in an immune response, for example by assaying for improvement of a physiological condition of a subject having a disease (such as a tumor). Effective amounts also can be determined through various in vitro, in vivo or in situ assays.

Therapeutic agents can be administered in a single dose, or in several doses, for example weekly, every 2 weeks, monthly, or bimonthly, during a course of treatment. However, the effective amount of can be dependent on the source applied, the subject being treated, the severity and type of the condition being treated, and the manner of administration.

In one example, it is an amount sufficient to partially or completely alleviate symptoms of a tumor in a subject. Treatment can involve only slowing the progression of the tumor temporarily, but can also include halting or reversing the progression of the tumor permanently. For example, a pharmaceutical preparation can decrease one or more symptoms of the tumor (such as the size of the tumor or the number of tumors or the number of metastases), for example decrease a symptom by at least 20%, at least 50%, at least 70%, at least 90%, at least 98%, or even at least 100%, as compared to an amount in the absence of the pharmaceutical preparation.

Treating a disease: “Treatment” refers to a therapeutic intervention that ameliorates a sign or symptom of a disease or pathological condition, such a sign or symptom of a tumor. Treatment can also induce remission or cure of a condition, such as a tumor. In particular examples, treatment includes preventing a disease, for example by inhibiting the full development of a disease, such as preventing development of a tumor (such as a metastasis). Prevention of a disease does not require a total absence of a tumor. For example, a decrease of at least 25% can be sufficient.

Tumor: A neoplasm. Includes solid and hematological tumors.

Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelogenous leukemia, and chronic lymphocytic leukemia), myelodysplastic syndrome, and myelodysplasia, polycythemia vera, lymphoma, (such as Hodgkin's disease, all forms of non-Hodgkin's lymphoma), multiple myeloma, Waldenstrom's macroglobulinemia, and heavy chain disease.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, lung cancer, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, menangioma, meningioma, neuroblastoma and retinoblastoma).

Tumor-associated antigen or tumor antigen (TAA): A tumor antigen which can stimulate tumor-specific T-cell-defined immune responses or antibodies to tumor cells. An immunogenic composition, such as a cancer vaccine, can include one or more TAAs. Particular examples are listed in Table 10.

Under conditions sufficient for: A phrase that is used to describe any environment that permits the desired activity.

In one example, includes administering a booster cancer vaccine and an agent that can deplete CD4 cells (such as a CD4 antibody) to a subject sufficient to allow the desired activity. In particular examples, the desired activity is enhancement of the effect of the vaccine.

Unit dose: A physically discrete unit containing a predetermined quantity of an active material calculated to individually or collectively produce a desired effect such as an immunogenic effect. A single unit dose or a plurality of unit doses can be used to provide the desired effect, such as an immunogenic effect.

Vaccine: An immunogenic composition that can be administered to a mammal, such as a human, to confer immunity, such as active immunity, to a disease or other pathological condition. Vaccines can be used prophylactically or therapeutically. Thus, vaccines can be used reduce the likelihood of developing a disease (such as a tumor or pathological infection) or to reduce the severity of symptoms of a disease or condition or limit the progression of the disease or condition (such as a tumor or a pathological infection).

A cancer vaccine is a vaccine that includes a therapeutic amount of one or more target tumor antigens, such as a vaccine that includes TAAs from a tumor of the: lung, prostate, ovary, breast, colon, cervix, liver, kidney, bone, or a melanoma. In one example, a cancer vaccine includes DRibbles or cells contacted with DRibbles. In a particular example, a vaccine includes the tumor present in the subject (e.g. an in situ vaccine).

An infectious agent vaccine is a vaccine that includes a therapeutic amount of one or more antigens specific for the infectious agent (such as a viral, bacterial, parasitic, or fungal peptide).

### Methods of Stimulating an Immune Response

Provided by this disclosure are methods of stimulating an immune response against a target antigen. In particular examples the target antigen is a tumor associated antigen (TAA), such as one or more tumor antigens expressed by a tumor cell. However, using the methods provided herein, one skilled in the art will appreciate that the methods can also be used with pathogenic antigens, such as viral, bacterial, or fungal antigens present in a vaccine. In particular examples, the methods manipulate regulatory T cells (T_{reg}) induced by cancer (iT_{reg}), so that anti-cancer vaccines can induce a strong tumor-specific T cell response. Without wishing to be bound to a particular theory, it is proposed that reducing or eliminating the tumor-induced T_{reg} (iT_{reg}) that limit anti-tumor immune responses without significantly deleting the natural T_{reg} (nT_{reg}) that prevent auto immune disease, can be used to enhance an immune response to a vaccine. Based on the results herein, it appears that one reason for the failure of tumor vaccines is that the regulatory T cells induced by the cancer or vaccination suppress the anti-tumor immune response. In particular examples, the methods increase tumor-specific T cell responses to represent at least 5% or at least 10% of the circulating T cell population, thereby improving clinical response rates.

For example, the methods can be used to stimulate an immune response in a subject against a tumor antigen, such as a mammalian subject (for example a human or veterinary subject) having a tumor that expresses the tumor antigen. Non-limiting tumors include benign tumors such as pituitary adenomas and gastrointestinal adenomatous polyps. Exemplary malignant tumors, include, but are not limited to: breast cancer, melanoma, lung cancer, renal cell carcinoma, prostate cancer, ovarian cancer, cervical cancer, colon cancer, liver cancer, or combinations thereof. Therefore, in particular examples the method is a method of stimulating an immune response against a tumor, such as a tumor expressing a tumor antigen. In some examples, the disclosed methods can be used to treat a subject having (or had) one or more tumors. For example, depletion of CD4+ T cells after administration of the first dose of an immunogenic composition can reduce one or more symptoms of a tumor, such as the size of a tumor, the number of tumors, or prevent metastasis of a tumor.

In particular examples, the method includes reducing or depleting the number of CD4+ T cells in a subject at a time subsequent to the subject receiving a first dose of a therapeutically effective amount of an immunogenic composition that includes one or more target antigens. Alternatively, if the vaccine is the tumor in situ, the method can include reducing or depleting the number of CD4+ T cells in a subject absent an exogenous administration of an immunogenic composition. For example, the CD4+ T cells can be depleted at least 10 days following administration of the first dose of the immunogenic composition, such as at least 14 days, at least 21 days, at least 30 days, at least 60 days, for example 10-14 or 10-21 days following administration of the first dose of the immunogenic composition. The subject is also administered a therapeutically effective amount of a second dose of the immunogenic composition, thereby stimulating an immune response against the target antigen. For example, if the target antigen is a tumor antigen, the subject can have a tumor that expresses one or more of the tumor antigens, or may have had such a tumor previously removed (for example surgically or chemically). In such examples, one or more tumor antigens expressed by a cell of the tumor are the same tumor antigens present in the immunogenic composition. For example, if the subject has or had breast cancer, the immunogenic composition includes one or more breast cancer TAAs.

In particular examples, the disclosed methods also include reconstituting the subject with PBMCs, such as autologus PBMCs. For example, the subject can be administered PBMCs prior to or at essentially the same time when the subject received the first dose of the immunogenic composition. In some examples, the PBMCs are significantly depleted of iT_{regs}, for example by depleting CD25+T cells, CD81+T cells, or both. Methods of depleting PBMCs of particular sub-populations of cells are known in the art, such as ex vivo methods. For example, the method can include incubation of the PBMCs with an antibody that recognizes a protein specific for iT_{regs}, such as an anti-CD25 or anti-CD81 antibody (or both), thereby permitting removal of cells which bind to the antibody. In some examples, the subject is lymphodepleted prior to administering the first dose of the immunogenic composition and the PBMCs, for example following apheresis of the subject. Methods of lymphodepleting a subject are known in the art.

In particular examples, the disclosed methods augment the immune response induced by the immunogenic compositions and more tumor cells are destroyed than if the immunogenic compositions were not used in combination with the disclosed methods.

In particular examples, the method can include significantly reducing the number of functional lymphocytes in the subject, prior to administration of a first dose of an immunogenic composition that includes one or more tumor antigens. For example, one or more lymphodepletion agents can be administered to the subject to reduce the number of functional lymphocytes present in the subject. In another or additional example, the method includes reconstituting the immune system of the lymphodepleted subject, for example by administration of functional lymphocytes previously obtained from the subject (such as PBMCs depleted of iT_{regs}, for example depleted of CD25+ cells, CD81+ cells, Areg+ cells, CD134+ cells, ptger3+ cells, or combinations thereof). In yet another example, the method includes obtaining blood cells from the subject prior to administration of a lymphodepletion agent. The method also includes administering to the subject a first dose of an immunogenic composition that includes one or more tumor antigens that are expressed by cells of the tumor and subsequently reducing the number of CD4+ T cells in a subject. Additional doses of the immunogenic composition are administered to the subject, for example administration of at least three doses of the immunogenic composition over a period of at least 180 days.

The disclosed methods can be used in combination with other therapies. For example, IL-2 and/or IL-12 can be administered during vaccination [100-700,000 IU/Kg IL-2]. In addition, T cell-directed co-stimulatory molecules like OX40R, 4-1BB or CTLA-4 can be stimulated by systemic administration of their recombinant ligands or by administration of specific monoclonal antibodies.

The disclosed method of anti-CD4 depletion can also be used alone, with the patients' tumor burden acting as the vaccine. This can also be used in combination with other therapies (such as IL2, anti-OX40R, anti-4-1BB, anti-CTLA-4, or combinations thereof).

### Depletion of CD4+ T Cells

The CD4+ T cells can be depleted in vivo or ex vivo. For example, for in vivo depletion, the method can include administering a therapeutically effective amount of one or more agents that significantly reduce the number of CD4+ T cells in the subject under conditions sufficient to reduce the number of CD4+ T cells in the subject. In some examples, the one or more agents that significantly deplete CD4+ T cells are administered simultaneously or nearly simultaneously (for example within 48 hours of each other, such as within 24 hours, within 6 hours, or within 1 hour) with the second dose of the immunogenic composition.

Exemplary agents that can be used to deplete CD4+ T cells include agents such as anti-CD4 antibodies, CD4 antisense molecules, and CD4 siRNAs. The therapeutically effective amount of the agents that significantly reduce the number of CD4+ T cells can be dependent on the subject being treated, the severity and type of the affliction, and the manner of administration. For example, a therapeutically effective amount of the agents that significantly reduce the number of CD4+ T cells can vary from an amount sufficient to decrease the number of CD4+ T cells in the subject by at least 20% to an amount sufficient to decrease the number of CD4+ T cells in the subject by at least 80%, such as an amount sufficient to decrease the number of CD4+ T cells in the subject by at least 30%, at least 50%, at least 70%, or at least 80%. In particular examples, CD4 cells are not completely eliminated, such that the regulatory (suppressive) activity is reduced but some helper activity is maintained. Methods of determining the number of CD4+ T cells in the subject are routine, thereby permitting a clinician to determine an appropriate dose of anti-CD4 (or other inhibitory agent). In addition, the exact amount can be readily determined by one of skill in the art based on the age, weight, sex, and physiological condition of the subject. Effective doses can be extrapolated from dose-response curves derived from animal model test systems or exploratory clinical trials. Those skilled in the art can determine an appropriate time and duration of therapy to achieve the desired effects on the subject.

### Immunogenic Compositions

The immunogenic compositions for use in the disclosed methods include therapeutically effective amounts of one or more target antigens, and can also include an immunostimulant, such as an adjuvant (for example CpG, MPL) or a cytokine, for example GM-CSF, or combinations thereof. In a particular example, the target antigen is a tumor antigen, such as an antigen expressed by a tumor present in a subject (or which has been removed from the subject). For example if the antigen is a tumor or tumor associated antigen, the immunogenic composition can be a cancer vaccine. Cancer vaccines can include whole tumor vaccines (autologous or allogenic) developed from cell lines, proteins or peptides overexpressed or specific for a tumor (such as Her2/neu and MUC-1), viruses or other vectors that encode TAAs (such as vaccinia, fowl pox virus, and plasmid DNA). Such vaccines are known in the art. Exemplary cancer vaccines include but are not limited to: ALVAC CEA B.71 (vaccine for colorectal cancer), ALVAC gp100M and Oncophage (vaccine for melanoma), Theratope (vaccine for breast cancer), Biovaxid® (vaccine for Follicular B-cell Non-Hodgkin's Lymphoma), GVAX™ (vaccine for prostate cancer), Oncophage (HSPPC-96, vaccine for kidney cancer), BEC2 (vaccine for lung cancer), as well as HPV vaccines for cervical cancer and others listed on the National Cancer Institute website. Exemplary lung cancer vaccines are listed in Ruttinger et al. (Current Immunotherapeutic Strategies in Lung Cancer, Surg. Clin. North Amer., 2007, in press). Parmiani et al. (J. Immunol. 178:1975-9, 2007) provides examples of unique tumor antigens that can be present in an immunogenic composition.

In a specific example, a cancer vaccine includes defective ribosomal products in blebs (DRibbles) or cells exposed to DRibbles. DRibbles can be produced and administered as a vaccine to a subject as described in WO 2007/016340. For example, a cell can be contacted with a proteasome inhibitor in an amount that does not substantially induce apoptosis of the cell, and under conditions sufficient for the cell to produce DRibbles. In some examples, the cells are also contacted with an amount of an agent that induces autophagy, for example rapamycin or culture media that starves the cells (such as HBSS media). In particular examples, the cells are also contacted with an amount of an agent that reduces glycosylation of proteins, for example tunicamycin, sufficient to enhance DRibble production in the presence of the proteasome inhibitor. The resulting treated tumor cells can be administered to the subject at a therapeutic dose, for example alone or in the presence of an adjuvant or other immunostimulatory agent, or an anti-tumor agent, thereby stimulating an immune response against one or more DRiPs. Alternatively, DRibbles are isolated from the treated tumor cells and administered to the subject at a therapeutic dose (for example 10 million cell equivalents of DRibbles), for example alone or in the presence of an adjuvant or other immunostimulatory agent, or an anti-tumor agent, thereby stimulating an immune response against one or more defective ribosomal products (DRiPs). In some examples, the resulting DRibbles are incubated with an APC obtained from peripheral blood mononuclear cells (PBMCs) from the subject under conditions sufficient for the APC to present one or more DRiPs, thereby generating DRibble-loaded APCs. The resulting DRibble-loaded APCs are administered to the subject at a therapeutic dose (for example 10 million DC cells loaded with 10 million cell equivalents of tumor DRibbles) (alone or in the presence of another therapeutic agent, such as an immunostimulatory agent or an anti-tumor agent), thereby stimulating an immune response against one or more DRiPs.

The therapeutically effective amount of the immunogenic composition, such as a cancer vaccine, can be dependent on the subject being treated, the severity and type of the affliction, and the manner of administration. For example, a therapeutically effective amount of the immunogenic composition can vary from an amount sufficient to stimulate the immune system in the subject by at least 20%, such as at least 50%, or at least 100% against the target antigen present in the immune composition. The exact amount of immunogenic composition can be readily determined by one of skill in the art based on the age, weight, sex, and physiological condition of the subject. Effective doses can be extrapolated from dose-response curves derived from in vitro or animal model test systems. Those skilled in the art can determine an appropriate time and duration of therapy to achieve the desired preventative or ameliorative effects on the immune pathology.

### Lymphodepletion and Reconstitution

In addition to the depletion of CD4+ T cells, the subject can be lymphodepleted and subsequently reconstituted, for example prior to depleting the CD4+ cells. However, lymphodepletion is not required.

For example, to increase the initial expansion and late persistence of tumor-reactive CTL and HTL, prior to administration of a first dose of an immunogenic composition (such as a cancer vaccine), subjects can be administered one or more agents, that alone, or in combination, substantially lymphodeplete the subject. The lymphodepletion agents are administered at therapeutically effective amounts under conditions sufficient to achieve lymphodepletion in the subject. In some examples, multiple doses of one or more lymphodepletion agents are administered, such as at least 2 or at least three doses. In a specific example, the one or more lymphodepletion agents are administered on three consecutive days. In particular examples, a subject is substantially lymphodepleted if the number of lymphocytes in the subject decreases by at least 50%, such as at least 75% or at least 90%, following administration of the lymphodepletion agent.

In one example, a lymphodepletion agent is an anti-tumor chemotherapeutic agent, such as one or more anti-tumor chemotherapeutic agents. Such agents and dosages are known, and can be selected by a treating physician depending on the subject to be treated. Examples of lymphodepletion agents include, but are not limited to fludarabine, cyclophosphamide, or combinations thereof. In a specific example, 350 mg/m² of cyclophosphamide is administered intravenously over 1 hour on three consecutive days.

In particular examples, the method further includes lymphodepleting subjects, followed by reconstituting the immune system of the subject. For example, prior to lymphodepletion and administration of an immunogenic composition, blood cells (such as monocytes and macrophages) are obtained from the subject, for example by using leukapheresis. The isolated cells can be frozen until a time appropriate for introducing the cells into the subject. For example, thawed lymphocytes (such as PBMCs, for example PBMCs depleted of CD25 or CD81 cells, or both) can be administered to the subject at the same time as the first dose of the immunogenic composition is administered, or shortly before or after administration of the first dose of the immunogenic composition. Such reconstitution of the immune system can in particular examples enhance stimulation of the immune system.

Lymphodepletion can be evaluated using many methods well known in the art. In one example, a white blood cell count (WBC) is used to determine the responsiveness of a subject's immune system. A WBC measures the number of white blood cells in a subject. Using methods well known in the art, the white blood cells in a subject's blood sample are separated from other blood cells and counted. Normal values of white blood cells are about 4,500 to about 10,000 white blood cells/μl. Lower numbers of white blood cells can be indicative of a state of lymphodepletion in the subject.

In another example, lymphodepletion in a subject is determined using a T lymphocyte count. Using methods well known in the art, the white blood cells in a subject's blood sample are separated from other blood cells. T lymphocytes are differentiated from other white blood cells using standard methods in the art, such as, for example, immunofluorescence or FACS. Reduced numbers of T cells, or a specific population of T cells, can be used as a measurement of lymphodepletion. A reduction in the number of T-cells, or in a specific population of T cells, compared to the number of T cells (or the number of cells in the specific population) prior to treatment can be used to indicate that lymphodepletion has been induced.

### Administration

Any mode of administration can be used for administering an immunogenic composition, agents that deplete CD4+ T cells, and other compositions (such as lymphodepletion agents) disclosed herein. Immunogenic compositions, agents that deplete CD4+ T cells, and other compositions are administered to a subject in therapeutically effective amounts. Those skilled in the art, such as a treating physician, can determine an appropriate route of administration. In one example, administration of an immunogenic composition is subcutaneous, intradermal, or i.p. In another example administration of a lymphodepletion agent is intravenous.

In particular examples, a therapeutically effective amount of an immunogenic composition is administered in at least two unit doses, such as at least three unit doses, at least four unit doses, at least five unit doses, such as 13 unit doses, over a period of at least 60 days, at least 90 days, at least 180 days, or at least 365 days.

### Kits

Also provided by the present disclosure are kits that include agents that can be used to stimulate an immune response, for example in the treatment of a tumor. In some examples, the kit includes one or more agents that can deplete CD4 cells (for example an anti-CD4 antibody, siRNA, or antisense molecule). The kit can also include agents that reduce iT_{regs}, such as and an anti-CD25 antibody or an anti-CD81 antibody, one or more immunogenic compositions (such as a cancer vaccine), one or more anti-neoplastic chemotherapeutic agents, or combinations thereof.

### Example 1

### Anti-CD4 Augments Vaccine Efficacy

This example describes a method to reduce T_{reg} cells using an anti-CD4 monoclonal antibody (mAb), administered at the time of the second and third vaccines.

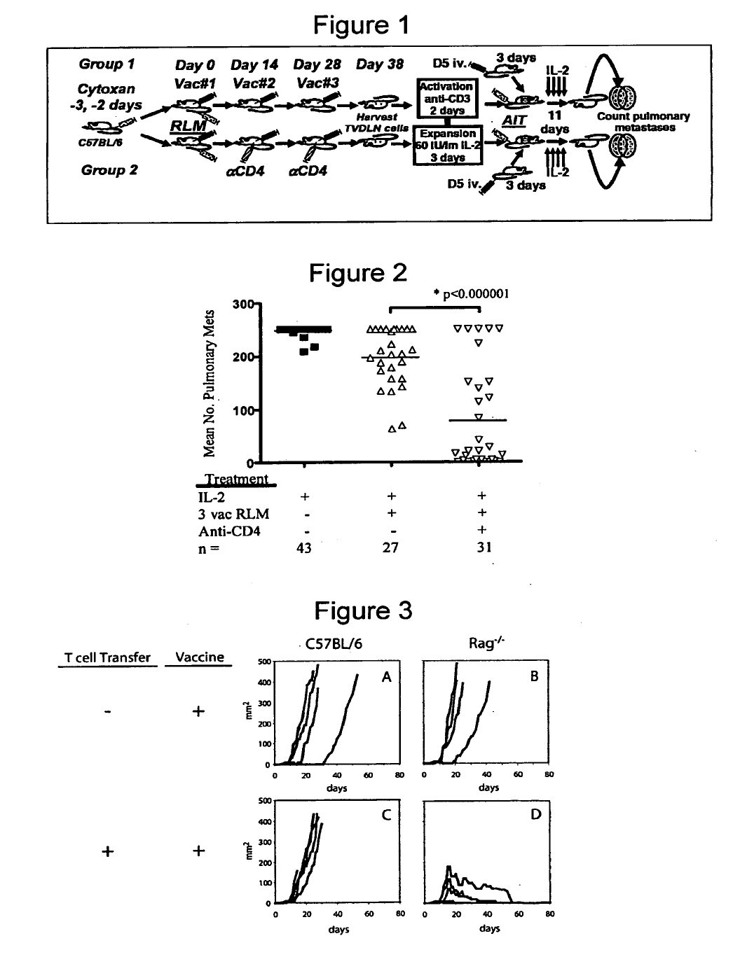

Mice (female wild-type C57BL/6 (H2b, Thy1.2+), 8-12 weeks of age, obtained from National Cancer Institute, Bethesda, Md.) were treated as shown in FIG. 1. Briefly, mice were made lymphopenic by intraperitoneal injection of cyclophosphamide (Cy, Bristol-Myers Squibb, Princeton, N.J.) at Cy200×2 (400 mg/kg Cy, q.d.) or Cy200×3 (600 mg/kg Cy, q.d.) on days −3 and −2. 24 hours later, treated mice were given 1 ml HBSS to assure ample urine output and preventing hemorrhagic cystitis caused by cyclophosphamide metabolites. 48 hours following the final cyclophosphamide treatment, mice were reconstituted with 2×10⁷ unfractionated splenocytes from naive C57BL/6 mice. Following reconstitution, mice were vaccinated with GM-CSF secreting B16BL6-D5 (D5) melanoma vaccine (s.c. injection of 2.5×10⁶ irradiated D5-G6 tumor cells) into each of the four flanks on days 0 and 14 and with 5×10⁵ irradiated or live tumor per flank on day 28. All mice were sacrificed 10 days later and spleens used for analysis and to generate effector T cells for ELISA and adoptive immunotherapy.

One group of RLM were depleted of CD4+ T cells by intraperitoneal injections of anti-CD4 (GK1.5) mAb 24 hours prior to second and third vaccinations (on days 14 and 28).

On day 38, spleens were harvested. Effector T cells were generated by using a standard protocol. Resulting spleens cells were activated for two days at 2×10⁶ cells/ml in complete medium (CM) in 24-well plates with 5 mg/ml 2c11 antibody (anti-CD3). T cells were harvested and expanded at 3-4×10⁵ cells/ml in CM containing 60 IU/ml IL-2 (Chiron Co., Emeryville, Calif.). in Lifecell tissue culture flasks (Nexell therapeutics Inc., CA) for three additional days. The resultant effector T cell population was used for the adoptive transfer and in vitro assays.

Effector T cells were transferred i.v. into B6 mice bearing 3-day pulmonary metastases established by tail vein injection of 2×10⁵ D5 tumor cells. The recipient mice received 90,000 IU IL-2 i.p. qd for 4 days starting from the day of T-cell transfer. Animals were sacrificed by CO₂ narcosis 13 days following D5 tumor inoculation and lungs were resected and fixed in Fekete's solution. Macroscopic metastases were enumerated. Lungs with metastases too numerous to count were designated as having 250 metastases.

FACS analysis of fresh splenocyte cells was performed as follows. Splenocyte cells were collected 10 days after vaccination and stained with different combinations of the following Abs purchased from BD Pharmingen (San Diego, Calif.) and eBioscience (San Diego, Calif.): FITC-CD4, PE-Cy7-CD3, and APC-CD8, PE, FoxP3, Cy-chrome-CD44, FTIC-1-Ab antibodies, PE-CD62L, PE-CD11c, FTIC-Ly6-C and Cy-chrome-CD8 antibodies. Purified anti-mouse Fc-receptor mAb, prepared from the culture supernatant of hybridoma 2.4G2 (ATCC, HB-197) was used to block non-specific binding to Fc receptors. Flow cytometric analysis was performed with the FACS Calibur and Cellquest software (Becton Dickinson, Mountain View, Calif.). At least 50,000 live cell events gated by scatter plots and through CD3 were analyzed for each sample.

Absolute PBL Counts were obtained as follows. Mice were sacrificed and bled through the eye orbital and collected into BD Vacutainer K2 EDTA tubes. Absolute lymphocyte counts were determined pipetting 100 μl of peripheral blood into a 4 ml facs tube and lysing red blood cells. The remaining lymphocytes were washed again and resuspended in FACS buffer and blocked with Fc receptor then stained for CD45, CD3, CD4, and CD8. Cells were washing again resuspended in 380 μl facs and 20 μl of Flow-Count fluorospheres (Beckman Coulter) were added to each tube. The percentages of CD3, CD4, CD8 lymphocytes and fluorospheres were determined by using a manually drawn lymphocyte scattergate. Absolute CD4 and CD8 T-cell counts were determined by using the ratio of CD3, CD4 or CD3, CD8 lymphocytes to fluorospheres counted using the following formula: cells per ul=[(cells counted)/(fluorospheres counted)]×fluorospheres/microliter×dilution factor (4).

IFN-γ ELISA was performed using effector T cells generated as described above. 2×10⁶ effector T cells were stimulated in vitro with 2×10⁵ cells D5 tumor cells, MCA-310 tumor cells, and D5 or MCA-310 cultured in 500 pg/ml recombinant IFN-γ for class-I up-regulation. T cells stimulated with plate-bound anti-CD3 antibody (10 μg/ml) or no stimulation were used as positive and negative controls. After culture for 24 hours, supernatant IFN-γ concentrations were determined by ELISA following manufacturer's protocols (Pharmingen). The concentration of IFN-γ was determined by regression analysis.

As shown in FIG. 2, mice vaccinated thrice (3 vac RLM), in the absence of other treatment, lost therapeutic efficacy. These results demonstrate the apparent development of potent T_{reg} cells following multiple vaccinations of reconstituted lymphopenic mice (RLM). These results demonstrate the development of T_{reg} cells following vaccination of reconstituted lymphopenic mice (RLM). However, this observation was not limited to RLM; non-lymphopenic “intact” mice (non RLM), vaccinated according to the same protocol, also exhibited a profound loss of therapeutic T cells. Therefore, vaccinations can induce T_{regs} that reduce or eliminate the beneficial effect of vaccination.

To overcome the T_{reg} cells, the ability of anti-CD4 to deplete T_{reg} cells was determined. As shown in FIG. 2, adding anti-CD4 treatment recovered significant (p<0.00001) antitumor activity. The increase in therapeutic efficacy was due to the relative decrease in T_{reg} CD4 T cells compared to the number of tumor specific CD8 T cells.

Multiply vaccinated animals also exhibited high frequencies of FoxP3+T cells, as detected by flow cytometry. Ten days following the third vaccine FOXP3⁺ CD4 T cells, while greatly reduced in absolute number (CD3⁺/CD4⁺/FOXP3⁺ cells were present at approximately 1/10 that of thrice vaccinated non CD4-depleted mice), were present at a percentage equivalent to thrice vaccinated non-CD4-depleted mice. Therefore, anti-CD4 did not preferentially deplete FoxP3⁺ CD4 T cells and T_{reg} cells were not selectively depleted. However, anti-CD4 substantially reduced the absolute number of CD4 T cells and T_{reg} cells.

### Example 2

### Immunotherapy Induces Regression of Large Established Poorly Immunogenic Tumors

This example describes methods used to demonstrate that use of immunotherapy reduces large established poorly immunogenic tumors in vivo.

Lymphopenic mice deficient of T_{reg} (Rag1−/−) were used to model the effect of T_{reg} depletion. Lymphopenic mice deficient of T_{reg} (Rag1−/−) or wild-type mice, each bearing 9 to 12 day s.c. induced B16BL6-D5 tumors, were used. Tumors continued to grow through the initiation of therapy and started to regress between day 15 and 19. Mice were vaccinated with D5-G6 (10⁷ cells, 10,000 R irradiated) s.c. on day 9 and received adoptive transfer of nayve tumor-specific CD4 and CD8 TCR transgenic T cells on day 9 adoptive transfer of effector tumor-specific CD4 and CD8 TCR transgenic T cells on day 12. Cells were administered intravenously. IL-2 was administered at 90,000 IU IL-2 per mouse i.p. on days 12-16, 18-22, 25-29.

As shown in FIG. 3, in the absence of T_{reg} cells (Rag1−/−), adoptive transfer of T cells and vaccination mediated regression of large (100-200 mm²) (FIG. 3, panel D). B16BL6-D5 tumors as large as 400 mm² were eliminated by this method. However, when the same method was used in intact (wt) mice that contain T_{reg}, the therapy was ineffective (FIG. 3, panel C). This therapeutic effect was also eliminated by the addition of CD4+ TCR Tg T cells that express FoxP3.

These results demonstrate that T cells and vaccines can mediate the elimination of large established poorly immunogenic tumors when T_{regs} are reduced (there were low numbers of FoxP3+ cells present in the T cells used).

### Example 3

Use of Anti-CD4 in Humans to Increase Vaccine Efficacy

Based upon the observations in Examples 1 and 2, a clinical trial is proposed for men with advanced hormone-refractory prostate cancer (HRPC). This example describes methods that can be used to treat HRPC using a prostate cancer vaccine in combination with an anti-CD4 antibody. Although methods for treating prostate cancer are particularly described, one skilled in the art will appreciate that similar methods can be used for other tumors, using an appropriate cancer vaccine (e.g. if the subject has breast cancer, a breast cancer vaccine is used instead of the prostate cancer vaccine described). In addition, variations in dosages or timing of administration can be made by a skilled clinician. Furthermore, one skilled in the art will appreciate the method may be practiced without the lymphodepletion step.

It was observed in men with HRPC who received cyclophosphamide prior to reconstitution and vaccination with Allogeneic Prostate GVAX® (Cell Genesys Inc.), or only vaccine, FoxP3+T cells rapidly recover in patients by the time of the second or third vaccine. This parallels the observation in the mouse model (FIG. 2). Therefore, it is expected that depletion of T_{regs} with anti-CD4 will enhance the immune response to the vaccine.

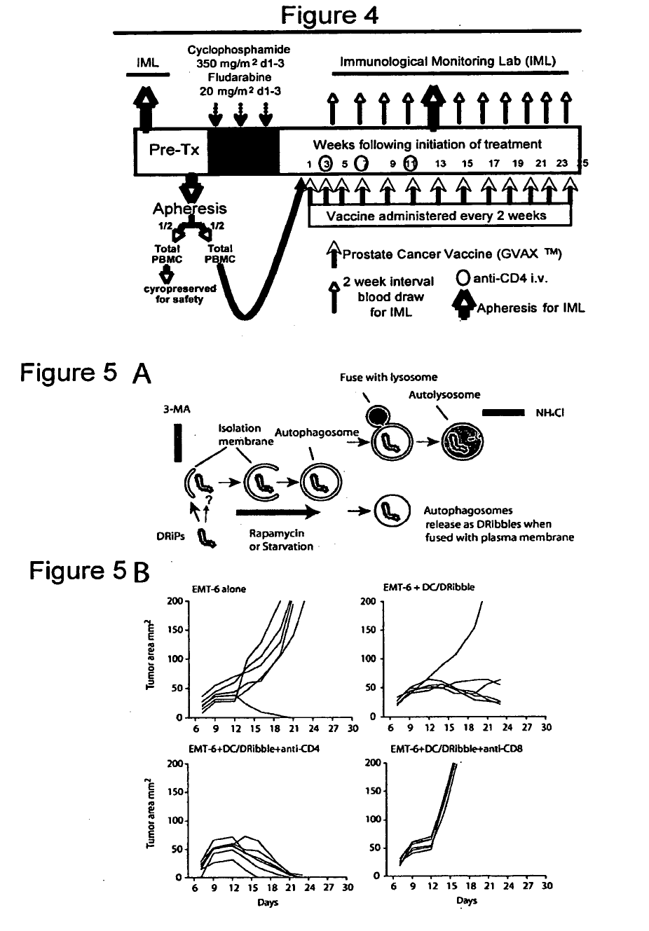

The method is outlined in FIG. 4. Subjects eligible for the trial include those with HRPC (including histologically diagnosed adenocarcinoma of the prostate and metastatic HRPC who have progressed despite one chemotherapy regimen).

PBMCs are collected using routine methods. Half of the apheresis product is unmanipulated and cryopreserved for later reinfusion if required. The remainder is cryopreserved for reinfusion following chemotherapy. In addition to apheresis for infusion, all subjects will undergo apheresis for collection of peripheral blood mononuclear cells (PBMC) for analysis of immune function. The first apheresis is done prior to vaccination with additional apheresis done at week 11 and 22. This procedure will be done over a minimum of 2 hours.

Men are made lymphopenic by treatment with chemotherapy (for example cyclophosphamide 350 mg/m² and fludarabine 20 mg/m² on days 1-3). Stored autologous PBMCs (2×10⁹-2×10¹⁰ cells) are reinfused i.v. on day 6.

Following the priming vaccination (5×10⁹ GVAX) at week 1 with Allogeneic Prostate GVAX® (Cell Genesys, Inc.), subjects will be observed in the clinic for 1 hour to assess for toxicity. Following the booster vaccinations (3×10⁹ GVAX every 2 weeks for 6 months), subjects will be observed in the clinic for 30 minutes or as clinically indicated. Prior to vaccination, local anesthesia can be administered with Emla cream (2.5% solution) at each vaccine site approximately 1 hour prior to injection as per manufacturer's instructions.

Men receive infusion of a monoclonal antibody to CD4 (HuMax-CD4, SAMerck Serono, Merck KGaA) at 1 mg/Kg i.v. at weeks 3, 7 and 11. Following the first i.v. infusion of HuMax-CD4 subjects will be observed in the clinic for 34 hours or as clinically indicated. It is anticipated that 1 mg/Kg will provide a CD4 depletion of between 30 and 80% of normal. The dosage of anti-CD4 can be adjusted to realize such a depletion. If after treating three patients this magnitude reduction in CD4 (and T_{reg} cells) is not realized, the dose of antibody can be increased (for example to 1.5 mg/Kg).

Lymphopenic patients reconstituted with PBMC, vaccinated, and administered anti-CD4 to reduce the absolute number of CD4 T cells. By reducing the number of CD4 T cells, the number of T_{reg} cells will also be reduced, for example by at least 30%, at least 50%, or at least 75%. If subjects develop grade III autoimmune disease they will be treated with high-dose steroids (for example 1000 mg hydrocortisone). If that is ineffective, subjects can be treated with a second cycle of cyclophosphamide and fludarabine and reconstituted with the other “half” of their apheresis.

PSA levels can be followed and slope calculated according to standard procedures. Bone scans and tumor measurements can be obtained in patients who have measurable disease using standard assessments.

The following methods can be used to detect prostate-specific T cell and B cell responses. DC are generated as previously described (Hu et al., J. Immunother. 27:48, 2004). Briefly, elutriated monocytes are cultured in X-Vivo 15 medium supplemented with 5% human AB serum, 1000 U/ml GM-CSF and 500 U/ml IL-4 for 7 days at 37° C. Harvested DC are typically greater than 90% CD11c+/HLA-DR+/lineage negative. PBMC, cryopreserved in human albumin, X-Vivo-15 and DMSO, is thawed, counted, and re-suspended in X-Vivo 15 medium and plated into 24 well plates that have been coated with anti-CD3 (10 ug/ml, Ortho OKT-3). Two days later activated T cells are harvested, counted, re-suspended at 10⁵ cells/ml in X-Vivo 15 medium containing 601u/mL-2 (Chiron) and plated into 6 well plates for 5 to 6 days culture with 5% CO₂ at 37° C. Effector T cells are harvested and assayed for functional activity against autologous DC transduced with control (GFP vector) or specific prostate antigen vectors.

Assays are used to determine the frequency of tumor-specific IFN-γ secreting T cells (for example using ICS), the amount of autololgous tumor-specific IFN-γ released (for example using ELISA), the frequency of autologous tumor-specific TNFα secreting T cells (for example using ICS), and tumor-specific expression of CD107 a/b. Supernatants can also be used to detect other cytokines released in response to specific tumor. ICS assays will counter stain with anti-CD3 and anti-CD4 or anti-CD8. This can be correlated with tumor-specific cytotoxicity detected in ⁵¹Cr-release assays.

Both pre and post apheresis samples can be analyzed for tumor-specific CD4⁺ and CD8 T cell responses.

It is expected that men receiving both the vaccine and the anti-CD4 will have a better prostate-specific immune response and will show a greater reduction in their tumor than men receiving only the vaccine (for example a reduction in tumor growth or tumor volume or a reduction in metastases).

### Example 4

### Treatment of Tumors Using Dribbles and Anti-CD4

This example describes methods that can be used to treat a tumor in vivo, using defective ribosomal products in blebs (DRibbles) derived from a tumor and anti-CD4. Although methods for treating breast cancer are particularly described, one skilled in the art will appreciate that similar methods can be used for other tumors, using DRibbles obtained from an appropriate tumor (e.g. if the subject has colon cancer, a DRibbles can be obtained from a colon cancer instead of the breast cancer DRibbles described). Alternatively, DRibbles containing proteins common to many tumors can be used across histologies (e.g. breast cancer DRibble vaccine can be used for a colon cancer vaccine, if the breast cancer and colon cancer have some similar tumor-associated proteins). In addition, variations in dosages or timing of administration can be made by a skilled clinician. Furthermore, one skilled in the art will appreciate the method may be practiced without the lymphodepletion step, or in combination with other methods (for example see Examples 6, 8-9 and 11).

DRibbles released from cells (such as tumor cells) after proteasome inhibitor-induced autophagy can accumulate defective ribosomal products (DRiPs) and short lived proteins (SLiPs) (and fragments thereof) in autophagy bodies and induce a strong immunity (such as anti-tumor) via cross-priming. As outlined in FIG. 5A, treatment of tumor cells with proteosome inhibition leads to the accumulation of antigens that are not typically cross-presented to the immune system (see WO 2007/016340, hereby incorporated by reference as to the method of making and administering DRibbles). The majority of autophagasomes accumulate inside tumor cells in the presence of NH₄Cl, a lysosomal inhibitor. Isolation of autophagasomes by sonication of “treated” tumor cells can improve recovery of antigens compared to the collection of “secreted” DRibbles recovered from culture supernatant.

Dendritic cells (DC) loaded with autophagasomes recovered from the supernatant of cultured tumor cells treated with proteasome inhibitor (DRibbles) induce tumor regression in mice bearing established breast cancer (FIG. 5B). As described above in Example and 2 and FIG. 3, effective elimination of T_{reg} cells, coupled with adoptive T cell transfer and vaccination, led to regression of large established poorly immunogenic tumors. These results demonstrate that T cells and vaccines can mediate the elimination of large established poorly immunogenic tumors when T_{regs} are reduced or eliminated. Inducing lymphopenia (creating “space”/decreasing Treg cells) enhanced therapeutic responses to vaccination. However, multiply vaccinated animals exhibited high frequencies of FoxP3+ T cells and loss of the therapeutic efficacy in adoptive immunotherapy studies (FIG. 2).

Therefore, methods that include administration of DRibbles in combination with anti-CD4 therapies (for example the anti-CD4 monoclonal antibody from Merck Serono), can be use to treat tumors. It is proposed that increasing cross-priming of tumor-specific T cells, while decreasing the suppressive effect of regulatory T cells, will improve the therapeutic efficacy of breast cancer (and other) vaccines.

Animal models of breast cancer are generated by administration of breast cancer cells (such as the cell lines EMT-6 and 4T1). Live EMT-6 tumor cells were injected into mammary glands of BALB/c mice. As shown in FIG. 5B, mice were either untreated (EMT-6), treated with dendritic cells loaded with DRibbles from EMT-6 cells (EMT-6+DC/DRibble), treated with dendritic cells loaded with DRibbles from EMT-6 cells and with anti-CD4 (EMT-6+DC/DRibble+anti-CD4), or treated with dendritic cells loaded with DRibbles from EMT-6 cells and with anti-CD8 (EMT-6+DC/DRibble+anti-CD8).

7-12 days after tumor injection, some mice were vaccinated with DC loaded with DRibbles from EMT-6 tumor cells on every other day for three injections and the fourth vaccination was given 14 days after the first s.c. vaccination. This vaccination schedule induces strong T-cell activation and expansion in vivo. Anti-CD4 (200 μg/dose, GK1.5, American Type Culture Collection (ATCC), Mannassas, Va.) or anti-CD8 (50 μg/dose, Ly5.2, ATCC) was administered on days 10 and 13 (one day before the second and third vaccination). The tumor was measured every other day whence they are palpable.

To measure immune responses to tumor cells, spleens can be harvested around 35 days after initial tumor injection at the time when control mice need to be sacrificed. Spleen cells are re-stimulated with DC loaded with DRibbles and expanded with IL-7 and IL-15 for 5 days. Activated T cells will be stimulated again with irradiated EMT-6, 4T1 tumor cells, and control syngenic 3T3 fibroblast to access their tumor-specific responses by measure the production of IFN-γ (for example using ELISA or intracellular staining and flow cytometric analysis).

As shown in FIG. 5B, DC loaded with DRibbles from breast cancer cells induced breast tumor-specific T cells and caused regression of established breast tumors (top right panel). However, this result was further enhanced by addition of anti-CD4 (FIG. 5, bottom left panel).

Similar methods can be performed in humans having breast cancer, for example using the CD80-modified MDA-MB-231 cell line to generate DRibbles in combination with anti-CD4 (for example using the dosage and administration regimen described in Examples 3 and 9-11), and in some examples also in combination with a method that depletes iTregs (for example depleting CD+25 T cells, CD+81 T cells, Areg+ T cells, or combinations thereof, as described in Examples 6 and 8-11). For example, breast tumor cells are treated with rapamycin to induce autophagy in the presence of inhibitors to both the proteasome and lysosome. Tumor cells are sonicated and DRibbles isolated. DRibbles are used to stimulate dendritic cells, which are administered to the subject (1-10×10⁶ DC with 1-100 cell equivalents of DRibbles administered s.c.) in combination with anti-CD4. Dendritic cells can be generated by culturing adherent PBMC with recombinant GM-CSF and IL-4 using methods well-known to those skilled in the art.

### Example 5

### Vaccination of RLM Reconstituted with Spleen Cells from Tumor-Bearing Mice (TBM) is not Effective

This example describes methods used to demonstrate that although vaccination of reconstituted-lymphopenic mice (RLM) significantly augments the development of anti-tumor T cells, use of spleen cells from tumor-bearing mice (TBM) is not effective.

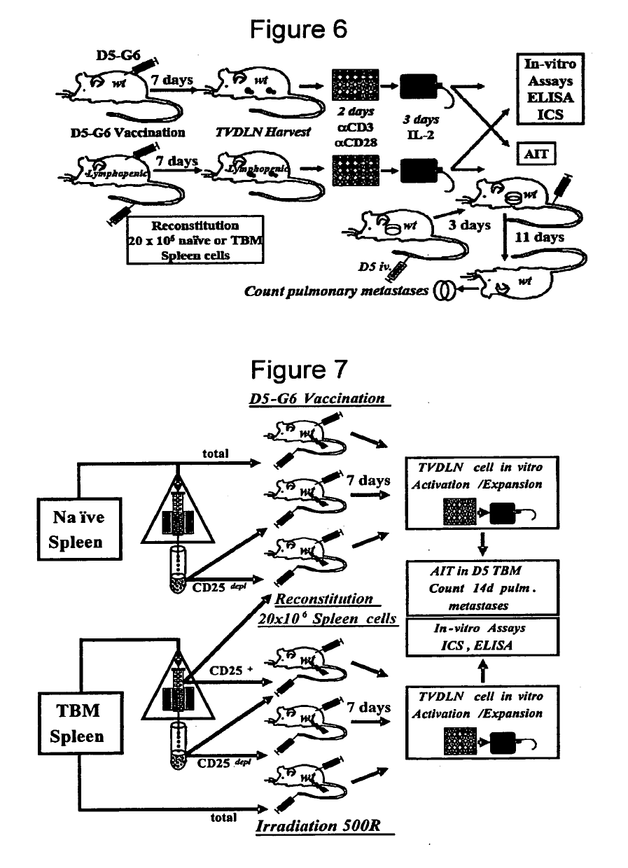

The basic experimental design is outlined in FIG. 6. Briefly, RLM (RAG-1−/− mice, lymphopenic) or wild-type were reconstituted with 20×10⁶ spleen cells from naïve or TBM animals, respectively, and immediately vaccinated with 10⁶ D5-G6 (GM-CSF secreting subclone of D5) tumor cells in all 4 flanks. On day 8 after vaccination, TVDLN were harvested and single cell suspensions stimulated for 2 days with soluble anti-CD3 (and in some examples also anti-CD28) in complete media (CM), washed and expanded in CM containing IL-2 (60 IU/ml) for 3 days. The resulting effector T cells (TE) were then washed and either adoptively transferred into B6 mice bearing 3-day-D5 pulmonary metastases or assayed in vitro for tumor-specific cytokine secretion by intracellular cytokine staining (ICS) and ELISA. Pulmonary metastases were evaluated 14 days after tumor inoculation.

Effector T cells (TE) generated in RLM not only contained higher frequencies of tumor-specific CD8⁺IFN-γ⁺ T cells, but also significantly (p<0.05) higher frequencies of tumor-specific CD4⁺IFN-γ⁺T cells. This tumor-specific CD4⁺ T cell response was determined by stimulation with D5 or MCA-310 tumor cells stably transfected with the CIITA transcriptional factor to express MHC class II. Coincident with the increased frequency of tumor-specific CD8⁺ and CD4⁺ TE adoptive transfer studies documented a significantly enhanced therapeutic efficacy (p<0.05) of RLM TE over TE generated in intact animals.

To determine whether reconstitution with T cells derived from a tumor-bearing mouse (TBM) would also be effective, lymphopenic mice reconstituted with spleen cells from TBM were vaccinated and resulting day-8 TVDLN were used to generate TE for in vitro analysis and adoptive transfer. Mice reconstituted with spleen cells from TBM were unable to generate tumor-specific T cells with therapeutic efficacy (Table 1). Therefore, effector T cells generated in RLM reconstituted with TBM spleens cells have lost the ability to secrete tumor-specific type-1 cytokines and can not mediate regression of established D5 pulmonary metastases upon adoptive transfer.

TABLE 1
Reconstitution with systemic tumor-bearing mouse (TBM) spleen cells
inhibits priming of therapeutic effector T cells.

| | | | Mean No. D5 pulm. Metastases | | | |
|---|---|---|---|---|---|---|
| RLM Donor | TE | IL-2 i.p. | Exp 1 | Exp 2 | Exp 3 | Exp 4 |
| None | None | + | >250 | >250 | >250 | >250 |
| Naïve | + | + | 0* | 0* | 24* | 32* |
| 8 d TBM | + | + | 99* | nd | nd | nd |
| 11 d TBM | + | + | nd | nd | >250 | >250 |
| 14 d TBM | + | + | nd | >250 | >250 | >250 |

To determine if depletion of CD4⁺ T cells from the TBM splenocytes used for reconstitution permits priming of tumor-specific TE in the RLM, the following methods were used. Spleen cells from TBM and naïve mice were harvested and single cell suspensions incubated with anti-CD4 beads (Miltenyi). CD4⁺ cells were depleted by passing the labeled spleen cells through a magnetic column. Negatively selected spleen cells from TBM and naïve mice were used to reconstitute lymphopenic mice (500R irradiation). Mice were then vaccinated with D5-G6 and TE generated from TVDLN cells were characterized for tumor-specific function in vitro and in vivo.

CD4 T cell depletion of TBM-derived spleen cells used in RLM did not lead to the recovery of priming of tumor-specific T cells. Furthermore, RLM reconstituted with CD4-depleted naïve spleen cells completely failed to prime tumor-specific T cells with therapeutic efficacy. Therefore, priming of tumor-specific TE in RLM is CD4 T cell-dependent.

### Example 6

### Depletion of TBM CD4⁺CD25⁺T Cells Recovers the Ability to Generate Tumor-Specific and Therapeutic TE

Spleen cells from 8-day TBM and naïve mice were evaluated by 8-color flow cytometry for a variety of surface activation markers. There was a >50% increase in the frequency of CD4⁺CD25⁺ T cells in spleens of TBM compared to T cells from naïve spleens. Based on this observation, this example describes methods used to demonstrate that depleting CD4⁺CD25⁺ cells from the spleen cells used for reconstitution would restore tumor-specific priming and TE-mediated therapeutic efficacy in the RLM model.

The general protocol is shown in FIG. 7. TBM spleen cells were depleted of CD25⁺ cells by a two-step antibody-magnetic bead process using a vario Macs column (Miltenyi). Single cell suspension of splenocytes from naïve or TBM are incubated with MACS anti-CD4 or anti-CD25 (anti-CD25 PE+anti-PE-bead) Micro Beads (Miltenyi Biotec, Calif.) for 25 minutes at 4° C. Stained cells are passed over a magnetic separation column in a Vario^{MACS} (Miltenyi) and the flow through containing cells depleted of specific subsets collected. Samples of cells are analyzed for purity of the separation by flow cytometry.

To enrich CD4⁺CD25⁺ T cells prior to flow cytometric sorting, single cell suspension of 3 spleens (≈300×10⁶) from naïve or TBM mice were incubated for 25 minutes at 4° C. with anti-CD8, anti-CD19 and anti-CD11b-MACS™ bead mAbs, washed and negatively selected via MACS™ magnetic column. Resulting cells (≈70×10⁶) were stained for 25 minutes at 4° C. with anti-CD4^{FITC} and anti-CD25^{PE}, washed and stained for 25 minutes at 4° C. with anti-PE-beads. After positive selection via magnetic column, an enrichment between 10-18% CD4⁺ CD25⁺ can be achieved, and after 45 minutes flow cytometric sorting of 0.5×10⁶ CD4⁺CD25⁺ or CD4⁺CD25⁻ T cells with a purity of >97.5% (viability >99%), cells can be used for further analysis or treatment. All assays are done in FBS-free condition using HBSS in 4-5 hours.

Naïve or TBM spleen cells were stained with anti-CD25 PE, incubated with anti-PE-magnetic beads (Miltenyi) and passaged over a vario Macs column to deplete CD25⁺ cells. Depletions were >98% effective. Total (non-depleted) or CD25-depleted spleen cells (20×10⁶ cells) from intact or TBM were used to reconstitute irradiated 500 R mice. Animals were vaccinated the same day with D5-G6 s.c., TVDLN harvested 8 days later, and the TE generated from TVDLN were used to measure cytokine (IFN-γ) release, ICS, and for adoptive transfer experiments (20×10⁶ effector T cells adoptively transferred into mice bearing 3-day pulmonary metastases). Supernatants after 24 hour tumor stimulation were analyzed by ELISA for the presence of IFN-γ release.

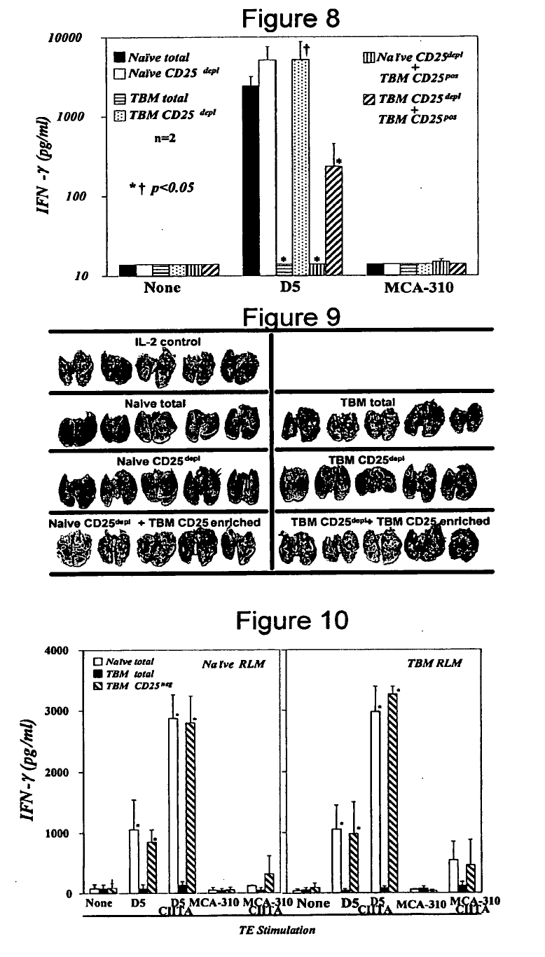

As shown in FIG. 8, depletion of CD25 cells from TBM spleen cells restored the tumor-specific IFN-γ response. Depletion of naïve CD25+ spleen cells does not enhance the tumor-specific response of TE generated in the RLM, but adding back TBM CD25+ spleen cells to the depleted populations eliminates priming again.

A similar strong recovery of therapeutic efficacy was seen for the effector T cells generated from RLM reconstituted with CD25-depleted TBM spleen cells (Table 2), indicating that iTreg (Treg population in TBM) do inhibit the priming of tumor-specific, therapeutic TE. Effector T cells generated from RLM reconstituted with total TBM spleen cells failed to mediate significant regression of pulmonary metastases in 3 of 4 experiments; however, when CD25⁺ cells were depleted from TBM spleen used in RLM, a significant reduction in pulmonary metastases was seen in all animals. This indicates that nTreg (Treg population in naïve mice) and do not inhibit priming upon vaccination with a GM-CSF secreting tumor vaccine in RLM.

TABLE 2
Depletion of CD25+ cells restores therapeutic efficacy generated in RLM

| | | | Mean No. of D5 pulm. metastases | | | |
|---|---|---|---|---|---|---|
| | | | (SEM)*, ^{†} , ^{♦} p < 0.05 | | | |
| RLM Donor | T_{E} | IL-2i.p. | Exp 1 | Exp 2 | Exp 3 | Exp 4 |
| None | None | + | >250 | >250 | >250 | >250 |
| Naïve total | + | + | 117 (98)* | 5 (4)* | 0* | 8 (8)* |
| Naïve CD25^{depl} | + | + | 193 (40)* | 7 (7)* | 0* | 42 (19)* |
| TBM total | + | + | >250 | 234 (25) | 188 (33)* | 233 (22) |
| TBM CD25^{depl} | + | + | 76 (67)*^{♦} | 50 (27)*^{♦} | 0*^{♦} | 22 (29)*^{♦} |
| Naïve CD25^{depl} + TBM CD25^{pos} | + | + | nd | nd | 43 (27)*^{†} | 196 (43)^{†} |
| TBM CD25^{depl} + TBM CD25^{pos} | + | + | nd | nd | 116 (29)*^{†} | 228 (24)^{†} |

Data shown represents the means of 4 consecutive experiments. The means (SEM) of 5 mice/group of D5 pulmonary metatsases.

In summary, while TE generated from RLM reconstituted with total TBM spleen cells failed to mediate significant regression of pulmonary metastases in 3 of 4 experiments, depletion of CD25⁺ cells from TBM spleen used in RLM resulted in a significant (p<0.05) reduction of pulmonary metastases in 4 out of 4 experiments.

To confirm that the CD25⁺ cells mediated this effect, CD25+ cells were “added back” to CD25-depleted populations of TBM or naïve spleen cells and subsequently used for reconstitution. The add back of CD25⁺ cells reduced (>90%) the tumor-specific IFN-γ release and therapeutic efficacy of effector T cells generated from RLM reconstituted with either CD25-depleted naïve or TBM spleens (FIGS. 8, 9 and Table 4). A photograph of lungs is shown in FIG. 9. The number of TBM CD4⁺CD25⁺ T cells added back normally present in the total TBM spleen cell population. Despite these lower numbers, significant suppression of anti-tumor activity was observed.

These results demonstrate that depletion of the CD25⁺ cells from TBM allows the remaining T cells to respond to vaccination and generate T cells with a tumor-specific IFN-γ profile and therapeutic efficacy in adoptive transfer studies.

To demonstrate that reconstitution of CD25-depleted TBM spleen cells is also be effective in lymphopenic mammals with an established tumor burden, the following methods were used. Lymphopenic (500R) 8-day TBM and naïve mice reconstituted with total naïve, total TBM, or CD25-depleted spleen cells (20×10⁶ cells) were vaccinated with D5-G6 (10⁶ tumor cells s.c.) (FIG. 7).

As shown in FIG. 10, reconstitution of tumor-bearing and naïve lymphopenic hosts with TBM spleen cells resulted in the complete suppression of tumor-specific TE IFN-γ responses. However, depletion of CD25⁺ T cells before reconstitution rescued the tumor-specific functional activity and therapeutic efficacy of the generated TE, even when the reconstituted lymphopenic hosts progressed in their tumor burden of the same immune suppressive melanoma (Table 3). The CD4+CD25+ T cells present in naïve mice existing prior to any treatment do not inhibit the generation of tumor-specific TE in RLM, and CD25-depletion of naïve spleen cells used for reconstitution in the RLM model does not further augment the priming of T cells with tumor-specific and therapeutic potential (Table 3).

TABLE 3
Depletion of TBM CD25+ restores therapeutic efficacy in TBM RLM

| | | | | Mean No. of D5 | |
|---|---|---|---|---|---|
| | | | | pulm. metastases | |
| RLM Donor | RLM host | TE | IL-2 i.p. | Exp 1 | Exp 2 |
| None | None | None | + | >250 | >250 |
| Naïve total | Naïve wt | + | + | 27 (21)* | 48 (23)* |
| D5 TBM total | Naïve wt | + | + | 238 (15) | 218 (30) |
| D5 TBMCD25^{depl} | Naïve wt | + | + | 25 (10)* | 32 (36)* |
| Naïve total | D5 TBM | + | + | 44 (20)* | 55 (7)* |
| D5 TBM total | D5 TBM | + | + | 235 (15) | 215 (42) |
| D5 TBMCD25^{depl} | D5 TBM | + | + | 28 (20)* | 40 (36)* |

Data shows the means (SEM) of 5 mice/group and D5 pulmonary metastases are counted 14 days after tumor inoculation and 11 days after adoptive transfer of TE.

### Example 7

### Phenotype of Tumor-Induced Regulatory T Cells (iTreg)

This example describes methods used to compare gene expression in CD4+CD25+ and CD4+CD25-subsets using gene microarray analysis.

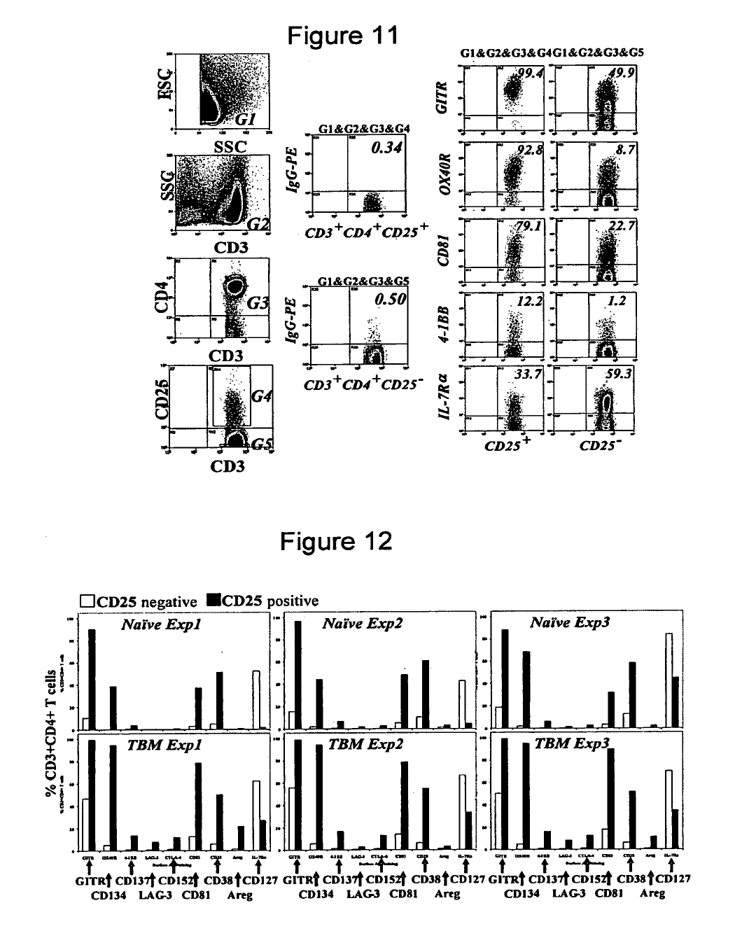

Pure subsets of CD4+ T cells were isolated from spleens of TBM and naïve mice. CD4⁺CD25⁺ (purity >99%) and CD4⁺CD25⁻ (purity >97%) spleen cells from 8-day TBM and naïve mice were sorted as indicated in FIG. 11. Spleen cells from TBM and naïve mice (3.37% CD4⁺CD25⁺ T cells) were magnetically depleted with anti-CD19, anti-CD8 and anti-CD11b beads to enrich for CD4⁺ T cells (from 19% to 87%; 10.8% CD4⁺CD25⁺ T cells). These pre-sorted cells were stained with anti-CD4FITC and anti-CD25PE and sorted using flow cytometry.

RNA was isolated (Qiagen RNAeasy) from the purified 0.5-2×10⁶ TBM and naïve CD4⁺CD25⁺ and CD4⁺CD25⁻ T cell populations and analyzed on Affymetrix micro arrays MOE 430A 2.0 (OHSU Affymetrix Micro Array Core). By comparing TBM versus naïve CD4⁺CD25⁺ and CD4⁺CD25⁻ spleen-derived T cells, indicator genes for CD4⁺CD25⁺ versus CD4⁺CD25⁻ T cells were identified (Table 4a). For example, CD25 expression was increased 28-30 fold, Foxp3 expression was increased 16-20 fold, GITR was increased 3-4 fold, and IL-2 expression was decreased 2-16 fold. A number of genes coding for receptors, ligands and soluble molecules important for cell-cell interactions also showed differential gene expression.

TABLE 4a
Gene Expression Ratio CD4+CD25+ vs. CD4+CD25−
8-day TBM and Naïve Spleen cells

| | | | | Naive |
|---|---|---|---|---|
| Gene | TBM Exp1 | TBM Exp2 | Naive Exp1 | Exp2 |
| CD25 | 29.86 | 27.86 | 36.76 | 48.50 |
| Foxp3 | 19.7 | 16 | 18.38 | 21.11 |
| GITR | 4.29 | 3.48 | 3.73 | 4.59 |
| CD134 (OX40R) | 6.96 | 5.28 | 5.66 | 8.57 |
| CD81 | 6.29 | 5.03 | 2.83 | 4.00 |
| CD83 | 8.57 | 6.5 | 6.50 | 6.96 |
| CD152 (CTLA-4) | 4.29 | 3.25 | 3.03 | 3.25 |
| CD137 (4-1BB) | 3.48 | 4.92 | 5.28 | 4.59 |
| Areg | 12.13 | 45.25 | 4.59 | 27.86 |
| Ptger3 | 12.13 | 36.76 | 3.73 | 3.73 |
| TNF-β (Lt-α) | 3.03 | 3.25 | 3.25 | 3.48 |
| CD120b (TNFRII) | 2.14 | 2.14 | 2.00 | 2.83 |
| CD38 | 2 | 3.03 | 2.64 | 2.83 |
| IL-2 | −2.3 | −16 | −2.52 | −3.73 |
| IL7R | −2.46 | −2.46 | −2.64 | −3.25 |
| IL-21 | −2.64 | −3.48 | −2.30 | −3.03 |
| TGFβR2 | −5.28 | −2 | −2.52 | −2.23 |

Gene expression ratio comparing purified CD4+CD25+ vs. CD4+CD25− naïve and 8-day TBM spleen T cells. After flow cytometric purification, RNA was analyzed using the murine Affymetrix Gene Array.

Genes whose expression increased from 3 to 45-fold in the comparison of CD4⁺CD25⁺ versus CD4⁺CD25⁻ T cells were chosen for further analysis, including CD134 (OX40R), CD137 (4-1BB), CD152 (CTLA-4), CD81, amphiregulin (Areg) and prostaglandin receptor EP3 (ptger3). CD81, a tetraspanin and co-stimulatory receptor, is 5-6 fold increased in TBM CD4⁺CD25⁺ over TBM CD4⁺CD25⁻ T cells with only a 2-4 fold increase on naïve CD4⁺CD25⁺ T cells. A 12-45 fold (TBM) over a 4-28 fold increase (naïve) for amphiregulin (Areg) was observed. Prostaglandin receptor EP3 (ptger3), a receptor for prostaglandin E2 (PGE2) with a distinct signaling capacity from the more ubiquitously expresses receptors EP2 and EP4, showed a 12-37 fold increase in TBM over only a 3-4 fold increase in naïve CD4+CD25+ T cells.

The expression profile of most of these markers was confirmed by flow cytometry. Briefly, 8-day TBM and naïve spleen cells are depleted magnetically of B-cells and macrophages and the enriched T cell suspension was stained with Fcγ-Block, anti-CD3^{FITC}, anti-CD4^{APCCy7}, anti-CD8^{PETR} and anti-CD25^{APC}. Cells were washed and split to be stained with anti-CD38^{PECy5}, anti-CD127^{PECy7} and for one of the indicated antigens (labeled with PE). As shown in FIG. 11, 99.4% of the CD25+ cells express GITR, 92.8% express OX40R, 79.1% express CD81, 12.2% express 4-1BB, and 33.7% express IL-7R.

FIGS. 11 and 12 summarize three independent paired experiments and analyzes for CD3⁺CD4⁺CD25⁺ or CD3⁺CD4⁺CD25⁻ T cells. CD81 and CD134 appear to be up-regulated on TBM as compared to naïve CD4⁺CD25⁺ T cells. CD152 (CTLA-4) is also measurable on the surface of TBM T_{reg} versus naïve T_{reg}. Since CTLA-4 was barely observed on the surface of T cells until stimulation with antigen on activated APC, this detectable surface expression on resting TBM CD4⁺CD25⁺ T cells is remarkable. Intracellular analysis of CD152 revealed a frequency of >80% of the TBM CD4⁺CD25⁺ versus 34% of the TBM CD4+CD25⁻ T cells being CD152+ (44% naïve CD4⁺CD25⁺CD152⁺ versus 17% naïve CD4⁺CD25⁻ CD152⁺ spleen-derived T cells).

Amphiregulin (Areg) was detectable on the cell surface of CD4⁺CD25⁺TBM T cells. While CD137 (4-1BB) mRNA expression was upregulated on TBM CD25⁺ in comparison to TBM CD25⁻ cells, surface expression analysis revealed a much smaller subset of TBM CD4⁺CD25⁺ being CD137⁺ compared to CD81 or CD134 expression. No significant change in LAG-3 gene expression for either CD25⁺ or CD25⁻ cell types was observed, and was used as a control (FIG. 12). The disclosed gene micro array analysis demonstrated a substantial up-regulation of Foxp3 mRNA in CD4⁺CD25⁺ T cells and intracellular expression correlated with the expression of CD25 on CD4⁺CD25⁺ T cells.

Based on these observations, CD25⁺, CD134⁺, CD81⁺, GITR⁺, CD137⁺, CD152⁺, CD38⁺, CD83⁺ and CD223⁺ subsets were depleted individually using magnetic bead depletion from the same TBM spleen cell pool and the depleted spleen cells used for reconstitution of lymphopenic hosts, which were subsequently vaccinated with D5-G6. Controls included RLM reconstituted with naïve spleen cells. After 8 days TVDLN were harvested, activated and expanded and TE assayed for tumor-specific cytokine release and therapeutic efficacy. As shown in Table 4b, depletion of CD134⁺, CD81⁺ and GITR⁺ cell subsets were equally capable of restoring therapeutic efficacy, like CD25-depletion. There was no significant difference (*; p>0.05) in the therapeutic efficacy of all four depletions when compared to the efficacy of naïve reconstituted RLM. Depletion of CD83+, CD137+, CD152+, CD38+ or LAG-3+ subsets delivers only miner or no recovery of therapeutic TE.

TABLE 4b
Depletion of CD81+, CD134+ and GITR+ TBM cells restores
generation of therapeutic TE

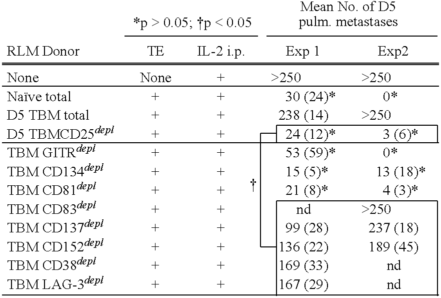

Data is shown as 2 consecutive experiments enumbering D5 pulmonary metastases from 5 mice/group after adoptive transfer.

These results also demonstrate that depletion of TBM CD83⁺, CD137⁺, CD152⁺, CD38⁺ and LAG-3⁺ T cells prior to reconstitution show significant reduction (†; p<0.05) in the therapeutic efficacy of TE. Tumor-specific cytokine release corresponds with the therapeutic efficacy of the different depleted subsets (FIG. 13). Further, depletion of cells bearing any of the markers was as efficient as depletion of CD25⁺ cells. Since 50% of CD4⁺CD25⁻ T cells also express GITR, depletion with this marker also reduced the total number of cells available after depletion for reconstitution by more than 70%, depletion of CD81⁺ and CD134⁺ T cells was comparable with depletion of CD25⁺ cells and reduced the total cell numbers for reconstitution by only 20%.

Therefore, these data indicate that CD81 and CD134 are markers for iTreg.

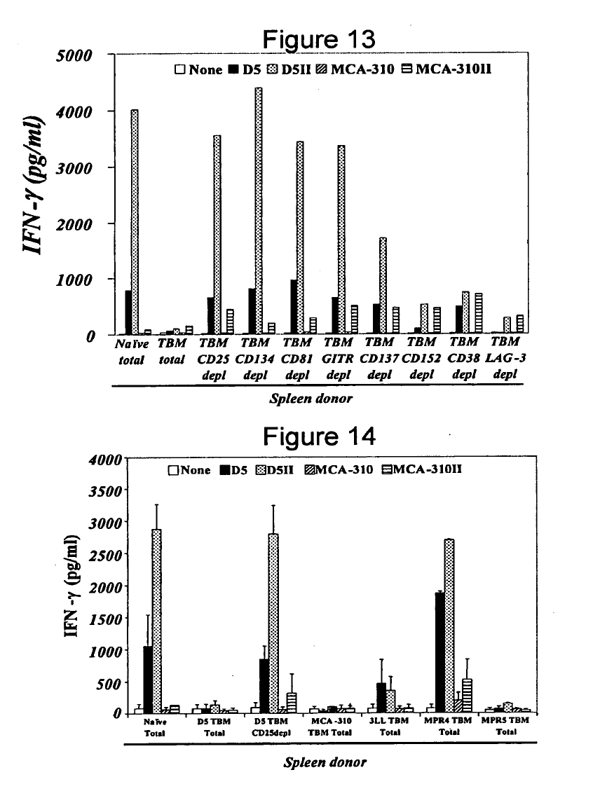

TBM spleen cells (8-day) from mice bearing the tumors indicated in FIG. 14 were used to reconstitute RLM vaccinated with D5-G6. Tumor-specific TE-mediated IFN-γ secretion was measured in 24 h tumor stimulation assays by ELISA. When effector T cells were generated from RLM mice that were reconstituted with spleen cells from mice bearing established syngeneic, but unrelated tumors (e.g. 3LL-lung carcinoma; MCA-310-sarcoma), partial to total suppression of tumor-specific (D5) T cell responses were observed. The observation was remarkable when spleen cells from mice with MPR4 or MPR5 were used in the RLM model. Both tumors are poorly immunogenic, but MPR5 was able to induce essentially complete suppression, while MPR4 did not induce suppression. Results of adoptive immunotherapy studies are summarized in Table 5 and in vitro function studies are summarized in FIG. 14.

TABLE 5
Tumor-induced suppression of therapeutic TE caused by
syngeneic, unrelated tumors

| | | | Mean No. of D5 pulm | |
|---|---|---|---|---|
| | | | metastases | |
| RLM Donor | TE | IL-2 i.p. | Exp 1 | Exp 2 |
| None | None | + | >250 | >250 |
| Naïve total | + | + | 27 (21)* | 48 (23)* |
| D5 TBM total | + | + | 238 (15) | 218 (30) |
| D5 TBMCD25^{depl} | + | + | 25 (10)* | 32 (36)* |
| MPR4 TBM total | + | + | 47 (34)* | 3 (2)* |
| MPR5 TBM total | + | + | 231 (19) | 202 (58) |
| MCA-310 TBM total | + | + | 212 (41) | 65 (16)* |
| 3LL TBM total | + | + | 193 (46) | 114 (33)* |

8-day TBM spleen cells from mice bearing the indicated tumors were used to reconstitute RLM vaccinated with D5-G6 and therapeutic efficacy was evaluated by the mean (SEM) regression of D5 pulmonary metastases in groups of 5 mice.

While depletion of D5TBM CD25+ spleen cells prior to reconstitution restores the generation of D5 tumor-specific TE, reconstitution in the RLM with either sarcoma MCA-310, lung carcinoma 3LL or in particular prostate carcinoma MPR5 TBM total spleen cells shows similar inhibition of the generation of D5 tumor-specific TE as reconstitution with D5 TBM total spleen cells when vaccinated with D5-G6. Reconstitution with MPR4 TBM total spleen cells did not suppress the generation of D5 tumor-specific, therapeutic T cells. TE generated in MPR4 TBM spleen-reconstituted RLM were equally therapeutic and functional as RLM reconstituted with naïve spleen cells. Since MCA-310 and 3LL both have more variable influence on their ability to suppress therapeutic efficacy, but reduce tumor-specific cytokine responses (Table 5), this indicates a secondary mechanism, such as inducing CD4⁺CD25⁺Treg cells. Since the most discussed suppressor molecules, TGF-β, prostaglandin E2 (PGE2) and IL-10 are highly expressed and TGF-β and PGE2 are secreted in vitro in substantial quantities on all tumors, other mechanism(s) may be responsible for the suppressive capabilities of MPR5 versus MPR4, MCA-310, 3LL or D5.

### Example 8

### Reducing Tumor-Induced Regulatory T Cells (iTreg)

As described above, four different markers (CD25, GITR, CD81 and CD134) identify iT_{reg} that inhibit the generation of therapeutic T cells. Areg and Ptgr3 are additional markers that are upregulated on iTreg. It is proposed that tumor-induced regulatory T cells (it_{reg}) are a subset of T_{reg} that can be selectively reduced or modulated resulting in the development of strong anti-tumor immune responses, without the elimination of nTreg that prevent autoimmune disease. Depletion of iT_{reg} ex vivo (for example depletion from a PBMC or TVDLN sample) or in vivo can be coupled with methods described herein to enhance an immune response against a vaccine, for example in combination with methods that deplete CD4 in vivo (for examples using anti-CD4 mAbs).

Methods that can be used are as follows. Eight to 12-week old female C57BL/6 (B6) mice from Jackson Laboratories (Bar Harbor, Me.) will be used unless otherwise noted. The D5 tumor is a clone of an early passage of B16BL6. This tumor is defined as poorly immunogenic, since immunization with 10⁷ 10,000R irradiated tumor cells does not protect against a minimal tumor challenge (2-5 times TB100). However, vaccination with 10⁷ D5-G6 (D5 stably secreting mGM-CSF) provides significant protection against tumor challenge. D5 is maintained in complete media. A large stock of D5 and D5-G6 tumor is cryo-preserved in liquid N₂ storage. At regular intervals new vials are thawed, established in culture and used for experiments. This practice has maintained a reproducible, poorly immunogenic tumor model.

D5-G6 tumor cells (10⁶) are injected subcutaneously in all four flanks of non-reconstituted, intact C57BL6 wt or lymphopenic mice (500cGy irradiated or Rag-1^{-/-}), that have been reconstituted with 20×10⁶ spleen cells harvested from naïve or 8-16 day systemic tumor-bearing mice (TBM). Eight days following reconstitution vaccination, TVDLN are harvested, stimulated with anti-CD3 for 2 days, expanded with IL-2 (60 IU/ml) for 3 days and the resulting effector T cells (TE) are adoptively transferred into wt mice bearing 3 day pulmonary metastases, established by i.v. injection of recipients with 0.2×10⁶ D5 tumor cells. Treated animals receive 90,000 IU IL-2 i.p. q.d. for 4 days. Mice are sacrificed 13 days following tumor inoculation by CO₂ narcosis. Lungs are resected, fixed in Fekete's solution and the number of pulmonary metastases evaluated. Alternatively, mice are followed for survival.

For intracellular cytokine analysis (ICS), 2×10⁶ TE from RLM and wt mice are stimulated for 12 hours in the presence of 5 μg/ml Brefeldin A (Sigma) in CM only (no stimulation), with 10⁵ specific tumor (D5), unrelated syngenic tumor (MCA-310), CIITA-stable-transfected D5-II and MCA-310-II with enhanced MHC class II expression or immobilized anti-CD3 in a 48 well plate. TE are harvested and stained with anti-CD8^{FITC} and anti-CD3^{PE-Cy5} mAbs, fixed/permeabilized in Cytofix/Cytoperm™ and stained intracellularly with anti-TNF-α^{PE} or anti-IFN-γ^{PE} mAb (Pharmingen, San Diego, Calif.). 50,000 gated CD8⁺/CD3⁺ TE are analyzed with a FACS™ Calibur and Cellquest software (Becton & Dickinson, San Diego, Calif., USA) or a CyAN and Summit/Winlist software (Dako Cytomation). Data is presented as the percentage of CD8⁺/CD3⁺/TNF-α⁺TE, or CD8⁺/CD3⁺/IFN-γ⁺TE, over the total number of CD8⁺/CD3⁺ TE. For ELISA, TE are stimulated and supernatants are harvested after 20-24 hours and tested for cytokines (IFN-γ) using commercially available reagents (Pharmingen). For 8-color/10-parameter flow cytometry analysis fresh spleen cells from wt and 8-day systemic D5 TBM are harvested, Fcγ receptor blocked (2.4G2 rat mAb; Pharmingen) for 20 minutes at 4° C., washed in FACS Buffer and stained in multiple 20 minute steps with anti-CD3^{FITC}, anti-CD4^{APC-Cy7}, anti-CD25^{APC}, anti-CD38^{PE-Cy5}, anti-CD127^{PE-cy7} and anti-CD134^{PE} or anti-GITR^{PE} or anti-CD81^{biotin/SA-PE} or anti-CD83^{PE} or anti-CD137^{PE} or anti-CD152^{PE}, (Pharmingen/eBioscience), anti-Foxp3^{PE or APC} (eBioscience, CA) and anti-CD8^{PE-TR} (CALTAG Lab., Burlingame, Calif.). 10-parameter flow cytometry analysis/sort on FCC/SCC/CD3⁺ cells was performed using a MoFlo cell sorter (Dako Cytomation, Fort Collins, Colo.) to compare naïve and TBM spleen cells. In order to set compensations and insure that the fidelity of staining is maintained when 8-color/10-parameter flow analysis is performed, controls are stained with anti-CD3^{FITC} only, or one other surface marker.

Magnetic bead separation can be performed as follows. Single cell suspension of splenocytes from naïve or TBM are incubated with MACS anti-CD4 or anti-CD25 (anti-CD25 PE+ anti-PE-bead) Micro Beads (Miltenyi Biotec, CA) for 25 minutes at 4° C. Stained cells will be passed over a magnetic separation column in a Vario^{MACS} (Miltenyi) and the flow through containing cells depleted of specific subsets collected. Samples of cells are always analyzed for purity of the separation by flow cytometry.

To enrich CD4⁺CD25⁺ T cells for time-reduced flow cytometric sorting, single cell suspension of 3 spleens (≈300×10⁶) from naïve or TBM mice were incubated for 25 minutes at 4° C. with anti-CD8, anti-CD19 and anti-CD11b-MACS™ bead mAbs, washed and negatively selected via MACS™ magnetic column. Resulting cells (≈70×10⁶) are now stained 25 min. at 4° C. with anti-CD4^{FITC} and anti-CD25^{PE}, washed and stained 25 min. at 4° C. with anti-PE-beads. After positive selection via magnetic column, an enrichment between 10-18% CD4⁺CD25⁺ can be achieved, and after 45 minutes flow cytometric sorting of 0.5×10⁶ CD4⁺CD25⁺ or CD4⁺ CD25⁻ T cells with a purity of >97.5% (viability >99%), cells can be used for further analysis or treatment. All assays are done in FBS-free condition using HBSS in 4-5 hours. For sorting an 8-color MoFlo (Dako Cytomation) and for analytical experiments a 9-color CyAN (Dako Cytomation) can be used.

The statistical significance of differences in numbers of metastatic nodules between experimental groups will be determined by the non-parametric, Wilcoxon rank-sum test. Two-sided p values <0.05 will be considered significant. All pulmonary metastases experiments are performed with T cells being transferred into groups of at least 5 mice. Survival analysis will be performed using Kaplan-Meier plots and Log rank sum tests using S-plus 2000© Software (Data Analysis Product Division, Mathsoft, Seattle, Wash.). The significance in cytokine release assays for multiple experiments will be assessed by a paired sample student's t-test.

Decreasing CD81 and CD134 Activity to Modulate Functional Activation of iTreg

As shown in Example 7, CD81, CD134, Areg, and Ptgr3 identify the tumor-induced regulatory T cells that inhibit the generation of therapeutic TE in the RLM model. Since both CD81 and CD134 are co-stimulatory receptors, stimulation and/or blockage via these receptors could modify either the development of iTreg in TBM or blockade of their regulatory function in the RLM model. Similarly Areg and Ptgr3 have signaling properties that may influence T_{reg} function or development. Thus, antibodies against these molecules/co-stimulatory receptors can be used to augment or reduce the function of iT_{reg} cells.

Treatment with anti-CD81 or anti-CD134 (or both) at the time tumor cells are injected into the donor mice can be used to prevent or reduce the generation of iT_{reg}, for example using the following methods. Mice will receive an i.p. injection of mAb (200 μg/day) specific for CD81 or CD134 (or both) on day 0, 3, 6, and 9 after systemic D5 tumor inoculation (2×10⁶ cells i.v.). TBM spleen is harvested on day 10, analyzed phenotypically for the frequency and number of iTreg and also assayed in the RLM model to determine if they inhibit the generation of tumor-specific (in vitro) and therapeutic TE (in vivo, adoptive transfer).

To determine whether anti-CD81 or anti-CD134 (or both) administered at the time of reconstitution and vaccination (day 0), day 3 and 6 post vaccination can prevent the iTreg from suppressing the generation of tumor-specific TE, the following methods can be used. Anti-GITR mAb treatment will be included as positive control. Rat IG will be administered as a negative control. Untreated TBM will serve as the negative control and CD25-depleted TBM spleen cells will be the positive control.

A multi color flow cytometry protocol can be used to investigate CD4⁺CD25⁺Foxp3⁺ T cells using antibodies to CD134, CD81, CD137 and GITR. To address whether the cytokine profiles of these Treg subsets are different, naïve and TBM spleen cells will be sorted to obtain subsets of CD134⁺CD81⁺, CD134-CD81⁻, CD134⁻ CD81⁺ and CD134⁺ CD81⁻. Expression of Foxp3/GFP can be determined and subsets of T cells would further be stimulated with immobilized anti-CD3 or PMA/Ionomycin with or w/o soluble anti-CD28 and secretion of IL-2, IL-4, IL-10, IL-13, IL-21 as compared to IFN-γ be determined by ELISA. Since CD4⁺CD25⁺ regulatory T cells secrete more type-2 cytokines and have reduced expression for IL-2 and IL-21 message, one or more of these cytokines could lead to a profile analysis that shows significant differences in the subsets of regulatory T cells described herein. CTLA-4 message was highly up-regulated in the gene micro array analysis and is highly intracellular expressed by CD4⁺CD25⁺ cells and even expressed in very small levels on their cell surface (FIG. 12). Therefore, TBM and naïve CD4⁺CD25⁺ Foxp3-GFP Tg T cells stained for CD81 and CD134 expression can also be analyzed for CTLA-4 expression.

It is expected that administration of anti-CD81 mAb, anti-CD134 mAb, or both, at the time tumor is administered to “initiate” the tumor-bearing state will reduce the development of iTreg or reduce the iTreg cells bearing CD81 or CD134. This will translate into an absence of iTreg in the TBM spleen and indicates that when these TBM spleen cells are used in vaccinated RLM, they will prime TVDLN T cells that will exhibit tumor-specific effector function that will be measured in the in vitro and in vivo assays specified above. Similarly, administration of anti-CD81 or anti-CD134 mAb to vaccinated TBM-RLM will likely restore anti-tumor function, which will be determined by the recovery of tumor-specific T cells from vaccinated animals (identified by ELISA or ICS), and recovery of therapeutic efficacy in adoptive transfer studies.

Decreasing Amphiregulin Activity to Modulate Functional Activation of iT_{reg}

It is shown in Example 7 above that amphiregulin (Areg) message is up-regulated in TBM CD4⁺CD25⁺ T cells. Areg secretion or expression on tumor-induced regulatory T cells may provide a signal in the tumor micro-environment to reduce T cell-mediated apoptosis of the targeted tumor cells. To determine whether treatment of naïve or TBM RLM with anti-Areg pAb (200 μg/d; LabVision Corp., Fremont, Calif.) or anti-EGFR mAb during vaccination affects the priming of tumor specific T cells with therapeutic efficacy, the following methods can be used. Clinical grade EGFR-kinase inhibitors (e.g. Cetuximab, Gefitinib and Erlotinib) can be used to suppress EGFR function on the tumor cells and in the mice in vivo. 500 cGy irradiated B6 (Thy1.2) or RAG-1^{-/-}-lymphopenic mice are reconstituted with naïve or TBM spleen cells and immediately vaccinated in all 4 flanks with D5-G6 as described above. Reconstitution with spleen cells from Areg^{-/-} (100) TBM, Areg^{-/-} naïve and wt naïve mice is used as a control. It is expected that treatment with anti-Areg will reverse iTreg function and promote recovery of tumor-specific therapeutic T cells.

To demonstrate how EGFR or Areg blockade affects the function of tumor-induced Treg, the following methods can be used. TBM, naïve and Foxp3-GFP Tg CD4⁺CD25⁺ and CD4⁺CD25⁻ T cells from control and treated animals are phenotyped and evaluated for changes in frequency or absolute number. They will then be purified by flow cytometry (MoFlo) and stimulated with immobilized anti-CD3 or PMA/Ionomycin with or without soluble anti-CD28 and in the presence or absence of neutralizing anti-Areg pAb for 12-48 hours. Supernatants will be assayed for Areg secretion (for example by ELISA or Western blot). In parallel, cells will be screened for intracellular Areg and Foxp3 expression (for example by flow cytometry). Sorted T cells can be stimulated with class II positive tumor cell lines, (D5, MCA-310, MPR5, MPR4, and 3LL; and all transfected with the CIITA construct for up regulation of 1-A^{b} (see Section C FIG. 2)) and culture supernatants assayed for cytokines and Areg as described above. In parallel, the same tumor cell lines can be exposed to recombinant Areg and culture supernatants tested for secretion of PGE2. The ability of Areg stimulation to induce tumor cells to secrete TGF-β or IL-10, two other Treg promoting cytokines, can be determined to evaluate whether Areg secreted by an iTreg in the tumor environment can stimulate tumor cells to further increase the immune suppression.

It is expected that the inhibition of EGFR signaling will reduce the number and function of iTreg T cells and lead to recovery of tumor-specific effector T cells when TBM spleen are used in RLM (TBM RLM). The in vitro assays will show that Areg secretion provides a signal in a regulatory “loop”, where iTreg that are induced by tumor-derived factors (TGF-β, PGE2) secrete Areg that binds EGFR on tumor cells and induces and/or augments their secretion of PGE2 and possibly other “pro” regulatory molecules. This could further promote or augment the stimulation of tumor-induced regulatory T cells.

To counter the possibility of Areg binding to EGFR on the tumor cells, tumor cells can be pre-incubate with anti-EGFR or anti-EGFR can be included in the T cell tumor stimulated culture.

Decreasing Prostaglandin Receptor EP3 Activity to Modulate Activation of iTreg

As shown in Example 7 above, ptger3 is up-regulated in TBM CD4⁺CD25⁺ spleen cells with potent iTreg function. Signaling through Ptger3 is mediated by PGE2, which is secreted by a variety of tumor cell lines. Therefore, it is proposed that tumor induced iTreg generation and function will be significantly reduced in mammals treated with COX2 inhibitor and in prostaglandin receptor EP3 (ptger3) knock out animals.

To demonstrate that reducing or blocking PGE2 signaling in RLM reconstituted with TBM spleen containing iTreg will overcome iTreg-mediated suppression and generate functional, and therapeutic tumor-specific T cells, the following methods can be used. Cox-2 inhibitor (SC58236; 3-10 mg/kg, Cayman Chemicals) or anti-PGE2-mAb (10 mg/kg; Cayman Chemicals) is administered i.p. on day 0 of reconstitution and 3 times/week from the starting day; PBS or IgG serve as a control. RLM mice are reconstituted with naïve or 10-16 day TBM spleen cells and vaccinated with D5-G6 as described above. TE are generated and tested for tumor-specific function in vitro and in vivo as described above. Spleen cells from ptger3^{-/-} can be used as controls.

Multiple treatments can also be combined. For example, TBM spleen cells from a ptger3^{-/-} mouse can be used to reconstitute a lymphopenic mouse vaccinated with D5-G6 and TNFRII:Fc, anti-CD81, anti CD134 and anti-Areg administered on day 0, 3 and 6. TVDLN are harvested on day 8 and resulting TE assayed for tumor-specific cytokine release in vitro and therapeutic efficacy.

To verify the impact of PGE2 on tumor-induced regulatory T cells, purified TBM, naïve and Foxp3-GFP Tg CD4⁺CD25⁺ and CD4⁺CD25⁻ T cells can first be stimulated with immobilized anti-CD3 or PMA/Ionomycin with or without soluble anti-CD28 or stimulated with tumor (D5, MCA-310, MPR5, MPR4, 3LL) or their culture supernatants for 12-48 hrs. Supernatants are screened for PGE2, Areg, TGF-β and IL-10 secretion (for example by ELISA). TBM, naïve and Foxp3-GFP Tg CD4⁺CD25⁺ and CD4⁺CD25⁻ T cells are further stimulated with the tumor cell lines above in the presence of Cox-2 inhibitor or anti-ptger3 pAb. T cells are harvested after 8-24 hrs and Foxp3 expression determined (for example by flow cytometry).

It is expected that preventing signaling of ptger3 on regulatory T cells directly by using the EP3R^{-/-} mouse and indirectly using COX2 inhibition will reduce their suppressive capacity and restore the generation of therapeutic, tumor-specific effector T cells. Changes in Foxp3 expression in the CD4⁺ T cell subpopulations indicate reduced activity of regulatory T cells. Expression of other markers of iTreg, such as CD81, CD134, and amphiregulin, will likely be affected.

Decreasing Lymphotoxin Alpha (LT-α) Activity to Moderate iTreg Suppressive Function

It is proposed that LT-α secretion by iTreg induces apoptosis of activated tumor-specific T cells. LT-α mRNA is up-regulated in TBM CD4⁺CD25⁺ T cells as compared to CD4⁺CD25⁻ T cells (see Example 7). Since LT-α can mediate apoptosis through TNFR and activated CD8⁺ T cells can up-regulate expression of TNFRI and TNFRII, secretion of LT-α by tumor-induced regulatory T cells may induce apoptosis of CD8 T cells responding to the tumor. Therefore, decreasing the activity of LT-α can be used to reduce or eliminate the ability iTreg to block priming or expansion of TE.

To demonstrate that reduction of LT-α activity can reduce iTreg-mediated suppression of tumor-specific immune responses in the TBM-RLM model, the following methods can be used. RLM are reconstituted with spleen cells from LT-α^{-/-} mice bearing 10-14 day systemic tumor (LT-α^{-/-} TBM) as described above. Only the reconstituting TBM spleen cells will be deficient in LT-α expression. As shown above in Table 3, it is the CD4⁺CD25⁺ T cells from this population that mediate suppression. Thus, if TBM spleen are obtained from a LT-α^{-/-} mouse and LT-α is a mediator, they should be unable to “suppress” the development of an anti-tumor immune response because they will be unable to induce apoptosis by secretion of LT-α. It is expected that the lack of LT-α in the iTreg generated from LT-α^{-/-} TBM will prevent the iTreg from eliminating activated CD8⁺ and CD4⁺ T cells. This will result in the generation of tumor-specific and therapeutic TE from RLM reconstituted with LT-α^{-/-} TBM spleen cells. Administration of TNFRII:Fc could yield the same result.

### Example 9

### Clinical Trial for Treatment of Hormone Refractory Prostate Cancer

This example describes methods used to deplete iTregs, which can be used to enhance vaccine efficacy. Although this example describes the use of particular agents to deplete Tregs, other agents can be used. For example, such methods can be used in combination with methods that deplete PBMCs of cells that express CD81, GITR, amphiregulin, ptger3, LT-α and CD134 as described above in Example 8. In addition, although prostate cancer is particularly described, one skilled in the art will appreciate that similar methods can be used for other cancers (for example by selecting an appropriate vaccine).

Although use of the GVAX vaccine is particularly described, one skilled in the art will appreciate that other hormone refractory prostate cancer (HRPC) vaccines can be used. Additional examples include sipuleucel (Provenge) (Small et al., J. Clin. Oncol., 24:3089-94, 2006), TRICOM (Dipaola et al., J. Transl. Med., 4:1, 2006), and BLP-25 (North et al., J. Urol., 176(1):91-5, 2006). Sipuleucel is prepared from autologous antigen-presenting cells loaded ex vivo with a prostatic acid phosphatase-GM-CSF fusion protein. TRICOM uses a prime and boost strategy with vaccinia (prime) and fowlpox (boost) virus engineered to produce PSA protein. BLP-25 is a liposome-encapsulated synthetic cancer mucin (MUC-1) vaccine.

Men receiving GVAX after chemotherapy to induce lymphopenia in an ongoing clinical trial were evaluated for the presence of T_{regs}. Men received cyclophosphamide (350 mg/m2×3d) prior to reconstitution and vaccination. Two weeks after the first vaccine, all six patients had CD3+/CD4+/CD25+/FOXP3+ T cells (Treg) at levels equal to their pretreatment samples. Four patients have had their week 11 apheresis characterized. All patients maintained their relative percentage of FOXP3+ T cells over the course of the analysis. Further, with the exception of one patient, who had a decrease in the absolute number of Treg cells, the remainder had a rapid recovery of their absolute CD4 counts and correspondingly, the absolute CD3+/CD4+/CD25+/FOXP3+Treg. Therefore, similar to the results described in Example 1 and shown in FIG. 2, vaccination can result in the development of detrimental T_{regs}. The apparent failure to deplete T_{reg} cells effectively using vaccine and lymphodepletion alone potentially limits the likelihood that vaccination will trigger the desired effect. The data support the rationale for the need to diminish T_{reg} as a component of any vaccination strategy.

Therefore, this example describes methods of depleting T_{regs}, for example by administration of anti-CD4, depleting CD25 cells, depleting CD81 cells, or combinations thereof, to treat a subject having cancer. For example, CD25 cells are significantly depleted from the pheresis product, CD4+ cells are depleted from the peripheral blood, or both. Although particular compounds are provided for achieving these results, the method is not limited to use of these particular compounds.

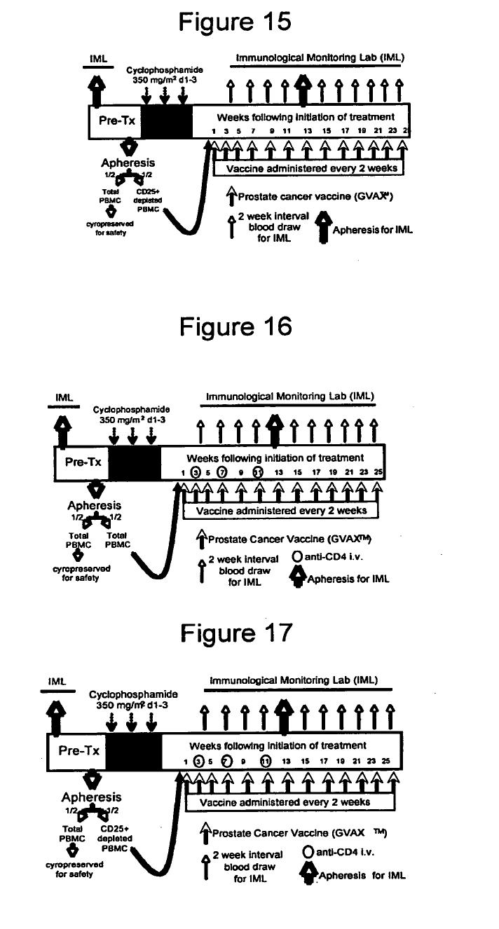

Tables 6a-c and FIGS. 15-17 describe the treatment regimen for each cohort. Individuals assigned to Cohort A will have CD25 depletion from pheresed PBMC, but will not receive anti-CD4 (zanolimumab). Subjects in Cohort B will receive autologous, unmanipulated PBMC and systemic doses of zanolimumab. Cohort C is a composite of the first two cohorts, and will use both ex vivo CD25 depletion of PBMC from a pheresis pack and in vivo CD4 depletion with systemic zanolimumab. Eligible patients will be randomly assigned to cohort to diminish selection bias that would complicate the interpretation of immunological findings.

TABLE 6a
Overview of treatment protocol

| | Apheresis for | Chemother- | | Anti-CD4 |
|---|---|---|---|---|
| Cohort | Reconstitution | apy | Vaccine | (zanolimumab) |
| A | CD25-depleted | CTX | GVAX q 2 wks | None |
| B | Total PBMC | CTX | GVAX q 2 wks | Wk 3, 7, 11 |
| C | CD25-depleted | CTX | GVAX q 2 wks | Wk 3, 7, 11 |

TABLE 6b
Cohort A. Patients Not Receiving anti-CD4 Antibody

| | | 5-10 days | | | | | | | | | | |
|---|---|---|---|---|---|---|---|---|---|---|---|---|
| | | before | Chemotherapy | Day | Day | Day 7: PRIME | Day 8, 10, | Week | Week | Week | Week | Week |
| | Screening | chemotherapy | Day 1-3 | 4 | 6 | VACCINATION | 12, 14 | 3 | 5 | 7 | 9 | 11 |
| Informed consent | X | | | | | | | | | | | |
| Physical exam/ | X | | X | | | X | | | X | | X | |
| medical history | | | | | | | | | | | | |
| ECOG and toxicity | X | | X | | | X | | X | X | X | X | X |
| assessments | | | | | | | | | | | | |
| Vaccinations | | | | | | X | | X⁴ | X⁴ | X⁴ | X⁴ | X⁴ |
| Leukapheresis for | X | | | | | | | | | | | |
| research purposes | | | | | | | | | | | | |
| Leukapheresis | | X^{2,8} | | | | | | | | | | |
| for reinfusion | | | | | | | | | | | | |
| Chemotherapy | | | X¹ | | | | | | | | | |
| Reinfusion of | | | | | X | | | | | | | |
| autologous | | | | | | | | | | | | |
| leukapheresis | | | | | | | | | | | | |
| product | | | | | | | | | | | | |
| CBC⁵ (w/5 part diff) | X | | X | X³ | X³ | X | X³ | X | X | X | X | |
| CMP | X | | X | | | X | | | X | | X | |
| CD4/CD8 | X | | | X | | | X | | | | | |
| Testosterone | X | | | | | | | | | | | |
| Hep B sAg, | | | | | | | | | | | | |
| Hep C Ab, | X | | | | | | | | | | | |
| HIV 1&2 Ab, HIV | | | | | | | | | | | | |
| RNA PCR | | | | | | | | | | | | |
| ABO, HTLV 1&2, | X | | | | | | | | | | | |
| RPR, Hep A Ab, | | | | | | | | | | | | |
| Hep B core Ab, | | | | | | | | | | | | |
| CMV | | | | | | | | | | | | |
| PSA | X | | | | | | | | | | X | |
| Immune parameters - | X | | | | | | | X | X | X | X | |
| 50 cc blood³ | | | | | | | | | | | | |
| Staging tests | X⁶ | | | | | | | | | | | |

| | Week | Week | Week | Week | Week | Week | Week | Week | Month | Long-term |
|---|---|---|---|---|---|---|---|---|---|---|
| | 12 | 13 | 15 | 17 | 19 | 21 | 23 | 25 | 7-12 | Follow-up |
| Informed consent | | | | | | | | | | |
| Physical exam/ | | X | | X | | X | | X | X | X |
| medical history | | | | | | | | | | |
| ECOG and toxicity | | X | X | X | X | X | X | X | X | X |
| assessments | | | | | | | | | | |
| Vaccinations | | X⁴ | X⁴ | X⁴ | X⁴ | X⁴ | X⁴ | X | | |
| Leukapheresis for | X | | | | | | | | | |
| research purposes | | | | | | | | | | |
| Leukapheresis | | | | | | | | | | |
| for reinfusion | | | | | | | | | | |
| Chemotherapy | | | | | | | | | | |
| Reinfusion of | | | | | | | | | | |
| autologous | | | | | | | | | | |
| leukapheresis | | | | | | | | | | |
| product | | | | | | | | | | |
| CBC⁵ (w/5 part diff) | X⁵ | X | | X | | X | | X³ | X | X³ |
| CMP | | X | | X | | X | | X | X | X³ |
| CD4/CD8 | | | | | | | | | | |
| Testosterone | | | | | | | | | | |
| Hep B sAg, | | | | | | | | | | |
| Hep C Ab, | | | | | | | | | | |
| HIV 1&2 Ab, HIV | | | | | | | | | | |
| RNA PCR | | | | | | | | | | |
| ABO, HTLV 1&2, | | | | | | | | | | |
| RPR, Hep A Ab, | | | | | | | | | | |
| Hep B core Ab, | | | | | | | | | | |
| CMV | | | | | | | | | | |
| PSA | | | | X | | | | X | X | X |
| Immune parameters - | | X | | X | | X | | X | X | X |
| 50 cc blood³ | | | | | | | | | | |
| Staging tests | | X⁶ | | | | | | X⁶ | X⁷ | X⁷ |

Cyclophosphamide 350 mg/m² IV daily, Days 1, 2, and 3.
The product is transported, processed and stored according to the American Red Cross Pacific Northwest Regional Blood Services policy.
To include an additional 4.0 ml purple-top tube sent to research lab for CD4 and CD8 T cell counts.
Boost vaccines are scheduled every 2 weeks +/− 3 days. Following each boost injection, patients will be observed in the clinic for 30 minutes, or as clinically indicated.
An additional CBC will be done within 24 hours prior to the leukapheresis per Red Cross standards (does not require 5 part diff).
Imaging to include bone scan and CT abdomen and pelvis.
Staging tests as clinically indicated.
Patients in Cohort A will have CD25 depletion of pheresis product before reinfusion.

TABLE 6c
Cohorts B and C. Patients Receiving anti-CD4 Antibody

| | | 5-10 days | | | | | | | | | | |
|---|---|---|---|---|---|---|---|---|---|---|---|---|
| | | before | Chemotherapy | Day | Day | Day 7: PRIME | Day 8, 10, | Week | Week | Week | Week | Week |
| | Screening | chemotherapy | Day 1-3 | 4 | 6 | VACCINATION | 12, 14 | 3 | 5 | 7 | 9 | 11 |
| Informed consent | X | | | | | | | | | | | |
| Physical exam/ | X | | X | | | X | | | X | | X | |
| medical history | | | | | | | | | | | | |
| ECOG and toxicity | X | | X | | | X | | X | X | X | X | X |
| assessments | | | | | | | | | | | | |
| Vaccinations | | | | | | X | | X⁴ | X⁴ | X⁴ | X⁴ | X⁴ |
| Leukapheresis for | X | | | | | | | | | | | |
| research purposes | | | | | | | | | | | | |
| Leukapheresis | | X^{2,9} | | | | | | | | | | |
| for reinfusion | | | | | | | | | | | | |
| Chemotherapy | | | X¹ | | | | | | | | | |
| Anti-CD4 | | | | | | | | X⁸ | | X⁸ | | X⁸ |
| Reinfusion of | | | | | X | | | | | | | |
| autologous | | | | | | | | | | | | |
| leukapheresis | | | | | | | | | | | | |
| product | | | | | | | | | | | | |
| CBC⁵ (w/5 part diff) | X | | X | X³ | X³ | X | X³ | X | X | X | X | |
| CMP | X | | X | | | X | | | X | | X | |
| Testosterone | X | | | | | | | | | | | |
| Hep B sAg, Hep C | X | | | | | | | | | | | |
| Ab, HIV 1&2 Ab, | | | | | | | | | | | | |
| HIVRNA PCR | | | | | | | | | | | | |
| ABO, HTLV 1&2, | X | | | | | | | | | | | |
| RPR, Hep A Ab, | | | | | | | | | | | | |
| Hep B core Ab, | | | | | | | | | | | | |
| CMV | | | | | | | | | | | | |
| PSA | X | | | | | | | | | | X | |
| Immune parameters - | X | | | | | | | X | X | X | X | |
| 50 cc blood³ | | | | | | | | | | | | |
| Staging tests | X⁶ | | | | | | | | | | | |

| | Week | Week | Week | Week | Week | Week | Week | Week | Month | Long-term |
|---|---|---|---|---|---|---|---|---|---|---|
| | 12 | 13 | 15 | 17 | 19 | 21 | 23 | 25 | 7-12 | Follow-up |
| Informed consent | | | | | | | | | | |
| Physical exam/ | | X | | X | | X | | X | X | X |
| medical history | | | | | | | | | | |
| ECOG and toxicity | | X | X | X | X | X | X | X | X | X |
| assessments | | | | | | | | | | |
| Vaccinations | | X⁴ | X⁴ | X⁴ | X⁴ | X⁴ | X⁴ | X | | |
| Leukapheresis for | X | | | | | | | | | |
| research purposes | | | | | | | | | | |
| Leukapheresis | | | | | | | | | | |
| for reinfusion | | | | | | | | | | |
| Chemotherapy | | | | | | | | | | |
| Anti-CD4 | | | | | | | | | | |
| Reinfusion of | | | | | | | | | | |
| autologous | | | | | | | | | | |
| leukapheresis | | | | | | | | | | |
| product | | | | | | | | | | |
| CBC⁵ (w/5 part diff) | X⁵ | X | | X | | X | | X³ | X | X |
| CMP | | X | | X | | X | | X | X | X |
| Testosterone | | | | | | | | | | |
| Hep B sAg, Hep C | | | | | | | | | | |
| Ab, HIV 1&2 Ab, | | | | | | | | | | |
| HIVRNA PCR | | | | | | | | | | |
| ABO, HTLV 1&2, | | | | | | | | | | |
| RPR, Hep A Ab, | | | | | | | | | | |
| Hep B core Ab, | | | | | | | | | | |
| CMV | | | | | | | | | | |
| PSA | | | | X | | | | X | X | X |
| Immune parameters - | | X | | X | | X | | X | X | X |
| 50 cc blood³ | | | | | | | | | | |
| Staging tests | | X⁶ | | | | | | X⁶ | X⁷ | X⁷ |

¹Cyclophosphamide 350 mg/m² IV daily, Days 1, 2, and 3.
²The product is transported, processed and stored according to the American Red Cross Pacific Northwest Regional Blood Services policy.
³To include an additional 4.0 ml purple-top tube sent to research lab for CD4 and CD8 T cell counts.
⁴Boost vaccines are scheduled every 2 weeks +/− 3 days. Following each boost injection, patients will be observed in the clinic for 30 minutes, or as clinically indicated.
⁵An additional CBC will be done within 24 hours prior to the leukapheresis per Red Cross standards (does not require 5 part diff).
⁶Imaging to include bone scan and CT abdomen and pelvis.
⁷Staging tests as clinically indicated.
⁸Zanolimumab, 1 mg/kg IV over no more than 30 minutes. Vital signs q 15 minutes × 4, then q 30 minutes × 2 after each dose.
⁹Patients in Cohort C will have CD25 depletion of pheresis product before reinfusion.

### Subjects

Members of all ethnic groups having histologically diagnosed adenocarcinoma of the prostate can receive the disclosed therapies. Patients with progressive HRPC as defined by radiographic progression of measurable or evaluable metastatic disease and/or a serial rise in two consecutive PSA concentrations taken over an interval of at least two weeks despite castrate (<50 μg/mL) levels of testosterone. Ideally, subjects have an ECOG performance status of 0 or 1, adequate bone marrow function (with the following parameters WBC≧3000/mm³; ANC≧1500/mm³; Untransfused Hgb≧9.0 g/dL; Untransfused Hct>28%; untransfused platelets>100,000/mm³), adequate renal function expressed by a serum creatinine less than 2.0 mg/dL, adequate hepatic function as evidenced by bilirubin≦2.0 mg/dL and ALT and AST≦1.5 times the upper limit of normal. Ideally, subjects have castrate levels of testosterone (<50 μg/mL).

Patients who have had radiation therapy as part of their initial treatment are eligible. Patients who have had palliative radiation therapy are also eligible if at least 28 days have passed since its completion and who have had less than 30% of their bone marrow treated. Ideally, patients have recovered from all side effects of their radiation therapy. Patients may have received one chemotherapy regimen for treatment of metastatic disease. Ideally, at least 28 days have elapsed since their last dose.

Subjects who can be excluded include those having transitional cell, small cell or squamous cell prostate carcinomas; those having had systemic steroid therapy within 10 days of enrollment; those having a history of active systemic lupus erythematosus, scleroderma, sarcoidosis, rheumatoid arthritis, ulcerative colitis, Crohn's colitis, glomerulonephritis or vasculitis; those having a clinically significant active infection; those having a history of other malignancies over the past five years (except for non-melanoma skin cancer or controlled superficial bladder cancer); those having uncontrolled medical problems (neurological, cardiovascular, or other illness); those having received prior treatment with an investigational drug within 30 days of study entry; those seropositive for HIV, hepatitis B surface antigen or hepatitis C; and those having clinical evidence of brain metastases or history of brain metastases.

### General Overview

Patients with metastatic hormone-refractory (e.g. androgen-independent) prostate cancer are selected. The general treatment course is as follows. Peripheral blood mononuclear cells (PBMC) are harvested between Days −5 and −10, and autologous transfer of CD25-depleted or total pheresis product on Day 6. Priming GVAX immunotherapy is administered on Day 7 followed by GVAX boosters every 2 weeks for 6 months. Lymphopenia induction is achieved by administering cyclophosphamide i.v. (350 mg/m²) on days 1-3. In some examples, patients are not lymphodepleted or asphersed prior to receiving the vaccine.

All subjects receive allogeneic prostate cancer vaccine Allogeneic Prostate GVAX® (Cell Genesys Inc.) according to the manufacturer's instructions. GVAX includes PC-3 and LNCaP allogeneic prostate tumor cell lines transduced with a retroviral vector containing the cDNA for the human GM-CSF gene. Subjects receive injections of CG871 to deliver a total dose of 1-3×10⁸ cells; injections of CG1940 to deliver a total dose of 1-3×10⁸ cells (total dose of all injections will be approximately 2-6×10⁸).

All subjects receive boost vaccinations (boost vaccinations will be administered every 2 weeks at a dose of 1-3×10⁸ CG8711 cells and 1-3×10⁸ CG1940 cells (to deliver a total dose of approximately 1-3×10⁸ cells). The total dose of all 12 vaccinations at the end of Week 25 for both cell lines will be approximately 2-5×10⁹ cells per patient.

Cohorts B and C will receive in vitro CD4 depletion. Patients will receive zanolimumab 1 mg/kg IV on weeks 3, 7 and 11, just prior to scheduled GVAX doses.

Cohorts A and C will receive a CD25 depletion of pheresis product. CD25 are depleted ex vivo from therapeutic leukapheresis using CliniMACSPlus Device, CliniMACS CD2S MicroBeads and associated reagents (Miltenyi Biotec) and Leukapheresis Transfer Bags and Connectors (Baxter/Fenwal).

### Vaccination

Allogeneic Prostate Cancer Vaccine (Cell Genesys, Inc.) is composed of two prostate cancer cell lines, PC-3 (CG1940) and LNCaP (CG8711), which have been genetically modified to secrete GM-CSF and are lethally irradiated to prevent cell division. Each “prime” dose includes approximately 2-6×10⁸ total cells, and each “boost” dose includes approximately 2-4×10⁸ total cells per boost.

Although particular dosages and timing of dosages are provided herein and by the manufacturer, a skilled clinician will appreciate that variations can be made without affecting the efficacy of the vaccine.

Prime vaccination will be administered on Day 7 deliver a total dose of approximately 1-3×10⁸ CG8711 cells and approximately 1-3×10⁸ CG1940 cells. The total dose of all injections will be approximately 3-6×10⁸ tumor cells. Following the Priming vaccination, patients can be monitored.

Booster vaccinations will be administered every 2 weeks (±3 days) to deliver a total dose of approximately 1-3×10⁸ CG8711 cells and a total dose of approximately 1-3×10⁸ CG1940 cells. The total dose of all 12 vaccinations at the end of Week 25 for both cell lines will be approximately 3-5×10⁹ cells per patient.

Prior to injection, local anesthesia with EMLA Cream (2.5% solution) can be applied at each vaccine site approximately 1 hour prior to injection PRN as per manufacturer's instructions. EMLA is an emulsion of acetamide, 2-(diethylamino)-N-(2,6-dimethylphenyl) 2.5% and propanamide, N-(2-methylphenyl)-2-(propylamino) 2.5% in which the oil phase is a eutectic mixture of lodicaine and prilocaine in a ratio of 1:1 by weight.

Pre- and post-vaccination immunity to CG1940 & CG8711 and PSMA can be assessed using assays that measure antigen-specific T cells using autologous dendritic cells to present freeze-thaw lysates of PC3 and LNCAP or recombinant PSMA protein. Soluble HLA-A2 can be used to present HLA-A2-binding peptides of PSMA and other prostate-associated antigens as they become available. Studies will be done to quantitate regulatory T cells (defined by expression of a CD4⁺CD25⁺ phenotype) and determine the level of FOXP3 expression (PCR and flow cytometry). Antibody titers to prostate antigens present in GVAX will be studied by ELISA. Cytokine production will be determined by bulk ELISA for IFN-γ, TNF-α, GM-CSF, IL-4, IL-5, and IL-10 as specified in the examples above using routine methods. Cytokine production for IFN-γ and TNF-α will be assessed by intracellular cytokine analysis using FACS. Repeated measures analyses will be performed on longitudinal data to assess patients' immune response profiles over time. Comparability of assay methods will be assessed with correlation analyses, regression analyses, standard parametric and nonparametric tests, and agreement methods.

### Leukapheresis/PBMC Collection for Autologous Transfer and Immune Monitoring

In particular examples, subjects undergo collection of peripheral blood mononuclear cells (PBMC) for future autologous transfer. Additional leukaphereses for immune monitoring can be done pre-treatment and Week 12. Leukapheresis for reinfusion purpose can be done with collection at 1 ml/min, at <3% colorgram, and over a minimum of 3 hours, with a goal of processing 12 liters. It will be taken to American Red Cross (ARC) for later autologous re-infusion according to ARC standard procedure for Hematopoietic Progenitor Cell processing, storage and re-infusion. A citrate solution is used in the machine to thin the patient's blood and prevent blood from clotting during the leukapheresis. Some of the citrate is given to the patient when blood is returned into the vein.

A baseline CBC can be done within 24 hours prior to the procedure. To proceed with collection, the results are ideally within the following parameters: WBC≧3000/mm³ and/or ANC≧1500/mm³; Hgb≧9.0 g/dL; Hct>28%; and platelet count≧100,000/mm³.

Patients can undergo additional leukapheresis collection 5-10 days prior to chemotherapy with subsequent reinfusion on Day 6.

In some examples, patients will undergo additional leukapheresis on week 12 (day 87) of their treatment. A CBC will be done within 24 hours prior to the leukapheresis procedure following ARC's standards procedures.

### CD25 Depletion

Patients in Cohorts A and C will have ex vivo depletion of CD25+ cells from the pheresis product using the CliniMACS column. Depletion of CD25 from PBMCs can be used to reduce T_{reg} cells so that the immune response to vaccination can be augmented. The resulting CD25-depleted PBMCs can be used to reconstitute lymphopenic patients (RLP) receiving a tumor vaccine. Using a CliniMACS separation device (Miltenyi, Inc.) and CD25 magnetic beads, an apheresis product was separated using the clinical grade reagents. Using all clinical grade reagents in a closed system, a 98.3% depletion of CD4⁺CD25⁺ cells from the apheresis product was obtained. Some subjects in this trial will have adoptive transfer of CD25-depleted pheresis product (Cohorts A and C), while others will have adoptive transfer of total PBMC (Cohort B).

The pheresis product will be split in half. One half will be cryopreserved and the remainder will undergo CD25 depletion as follows. The contents of one vial of CliniMACS CD25 MicroBeads is capable of labeling CD25 positive T cells out of a total leukocyte number of up to 40×10⁹ cells. The leukapheresis product is transferred into Cell Preparation Bag and the volume of leukapheresis product determined by weighing the empty and filled Cell Preparation Bag. A sterile sample is taken to determine total number of leukocytes and estimate a percentage of target cells and viability. The leukapheresis product is then diluted (1:3) with CliniMACS PBS/EDTA Buffer (supplemented with 0.5% HSA or BSA) and the cells centrifuged in the bag at 300 g for 15 minutes without brake. The supernatant is removed and the cell pellet is resuspended to a labeling volume of 95 mL for depletion. One vial CliniMACS CD25 MicroBeads is added sterile to the bag followed by mixing.

The cell preparation bag is incubated for 30 minutes at controlled room temperature (+19° C. to +25° C.) on an orbital shaker at 25 rpm. After incubation, buffer is added to a final volume of 600 mL for cell washing, and cells are spun down 15 minutes at room temperature and 300 g without brake. As much supernatant as possible is removed from the Cell Preparation Bag and cells are resuspended to a density of ≦0.4×10⁹ total cells/mL. The final sample volume of the leukapheresis product for loading on the CliniMACSPlus Instrument should not exceed 275 mL. The bag is connected sterile to the tubing set and placed into the Instrument along the programming.

Separation is fully automatic and generally takes 47 minutes. Final products are CD25-depleted and CD25-enriched cells in separate bags. The CD25-depleted product is transferred non-touched into Cryolite freezing bags after taking samples for flow cytometric phenotype and PCR analysis. The final CD25-depleted leukapheresis product undergoes cryopreservation along a controlled rate freezing program using a Cryomed Freezer (Therma Forma).

Cell samples from the total leukapheresis population and the CD25-depleted and CD25-enriched populations are analyzed for CD4+ and CD8+ T cell content as well as CD4+CD25+Foxp3+ expression. Total RNA is purified from all 3 populations for PCR analysis of FOXP3 expression. Viability and total cell counts are determined at all times.

### Chemotherapy

In some examples, subjects are lymphodepleted prior to administration of a vaccine. Although lymphodepletion is described in this example, a clinician will recognize that not all subjects will require lymphodepletion prior to administration of a cancer vaccine.

Patients who are lymphodepleted will receive cyclophosphamide (Cytoxan®, 2-[bis(2-chlorethyl)amino]tetrahydro-2H-1,3,2-oxazaphosphorine 2-oxide monohydrate) (NSC-26271) 350 mg/m² i.v. on Days 1, 2,3. In some examples, subjects are lymphodepleted using fludarabine 20 mg/m² i.v. on days 1, 2, 3. In some examples, both cyclophosphamide and fludarabine are used. Hydration and antiemetics (excluding dexamethasone or other steroids) can be used at the treating physician's discretion.

Cyclophosphamide is supplied in 100 mg, 200 mg, 500 mg, 1 gram and 2 gram vials as a white powder, and can be reconstituted with sterile water for Injection, USP, and can be diluted in either normal saline or D5W. The PO form of cyclophosphamide is supplied as 50 mg and 25 mg tablets. Cyclophosphamide can be administered for 3 consecutive days, for example starting on a Wednesday. Cyclophosphamide is diluted in about 150 cc of normal saline or D5W and infused intravenously over 30-60 minutes. An added dose of IV fluids may help prevent bladder toxicity.

Should an episode of febrile neutropenia occur, all subsequent patients in that cohort, and the next cohort if applicable, can receive prophylactic G-CSF according to standard practice.

### Autologous PBMC Reinfusion

Autologous PBMC product is reinfused on Day 6 as an outpatient.

### Anti-CD4 Administration

Using an anti-CD4 monoclonal antibody (mAb) permits significant reduction in CD4⁺/CD25⁺/FOXP3+T_{reg} cells in the peripheral blood. A CD4-depleting antibody (one example is zanolimumab) is administered at a dose of 1 mg/kg IV over no more than 30 minutes on weeks 3, 7, and 13 (or 11) on days of GVAX vaccination prior to the vaccination. Patients will be monitored with vital signs q 15 minutes×4, and then q 30 minutes×2 after each zanolimumab administration.

This dose of zanolimumab was chosen based on good tolerance (no grade 3 or greater toxicities) and the goal to decrease T_{reg}, but not eliminate all CD4 T cells. If adverse events from infection or prolonged immunosuppression, a lower dose of zanolimumab can be used (such as 0.1-0.5 mg/kg). Conversely, if T_{reg} are not depleted with zanolimumab at 1 mg/kg, then a higher dose (such as 1.5-3 mg/kg) can be used. In particular examples, the dose of zanolimumab used is one that reduces CD4 cells by at least 30%, at least 50%, or at least 75%, for example 30-90% or 30-80%. For example, zanolimumab can be used to decrease T_{reg}, but not eliminate all CD4 T cells.

Treatment with zanolimumab decreases the total lymphocyte count due to a depletion of CD4+ T-lymphocytes. Zanolimumab is not administered to subjects with a current serious infection including sepsis, or to subjects with previous severe hypersensitivity reactions to any of the components. Zanolimumab is a human immunoglobulin (IgG1κ) anti-CD4 monoclonal antibody which recognizes an epitope expressed on the membrane-bound CD4 molecule in a subset of T-lymphocytes and on human, cynomolgus and chimpanzee monocytes.

### Dose Interruptions and Management of Autoimmune Events

With the exception of treatment-related toxicities, dose interruptions for CG1940 and CG8711 are strongly discouraged. A subject who is unable to receive the scheduled dose of vaccine during the allotted timeframe will miss that scheduled dose. The subject will resume treatment at the next scheduled treatment visit.

Treatment may be restarted at the discretion of the clinician if the toxicity resolves to less than or equal to grade 2. If a treatment-related toxicity does not resolve to less than or equal to grade 2 in two weeks, the subject will not receive further treatment.

It is possible that patients may experience significant autoimmune phenomena due to suppression of T_{reg} from the CD4 modulating maneuvers in this method. If grade I or II autoimmune events occur (graded with CTCAE v3 criteria) then no dose interruptions or modifications will be made. If grade III or IV autoimmune events occur, then steroids and other supportive interventions will be administered. If the clinical manifestations of the autoimmune event do not improve to grade II or better after steroids, then subjects in Cohorts A and C who have had CD25 depletion will receive another cycle of cyclophosphamide (350 mg/m² IV daily×3) (or other lymphodepletion agent) followed by infusion of autologous cryopreserved total PBMC, which contain T_{reg}, with the goal of dampening the adverse immune response.

The definition for dose-limiting toxicity is grade III or greater non-hematologic toxicity. If grade III or greater non-hematologic treatment-related toxicity occurs in two patients in any cohort, then accrual to that cohort will cease. Accrual to other cohorts may continue.

### Screening

Thirty days prior to apheresis, the following clinical and laboratory evaluations can be performed. ECOG Performance Status; tumor assessment (radiographic assessment, as clinically indicated); PSA; immunological monitoring to include an additional 4.0 ml purple-top tube for CD4 and CD8 T cell counts; complete blood count (CBC), differential, platelets, hematocrit, hemoglobin, and 5-part differential; CD-/CD-8 count; chemistry including Sodium, potassium, chloride, BUN, creatinine, glucose, total protein, albumin, calcium, total bilirubin, alkaline phosphatase and AST; hepatitis B surface antigen and hepatitis C antibody; hepatitis A and hepatitis B core antibody; hepatitis sAg, hepatitis C antibody; ABO, HIV 1 & 2 Antibody, HIV 1 Ag (or HIV RNA PCR), HTLV 1 & 2, RPR, and CMV; and testosterone.

Patients can undergo leukapheresis for collection of mononuclear cells for immunological monitoring. Within 24 hours prior to leukapheresis, subjects can have a CBC and immune parameters drawn, including a 4.0 ml purple-top tube for CD4 and CD8 T cell counts.

Cyclophosphamide will be administered at a dose of 350 mg/m² intravenously over 1 hour on Days 1, 2 and 3 of the protocol. Routine antiemetics (with the exception of steroids) can be used for symptom management at the clinician's discretion.

### Vaccination and Anti-CD 4 Evaluations (Day 7 and Every 2 Weeks to Week 25)

Prior to administration of the vaccine or anti-CD4, the following assessments can be made. A clinical assessment to assess the patient for adverse events before each vaccination, including weight and vital signs, and evaluation of injection site reactions at all previous sites of vaccination. A physical exam (including ECOG Performance Status) can be performed, including weight and vital signs at least every four weeks, or more frequently as clinically indicated. PSA can be measured every 8 weeks during vaccination and as indicated thereafter. Immunological monitoring will be performed, as well as a CBC and Comprehensive Metabolic Panel (CMP).

Imaging studies can be performed every 3 months while on treatment and thereafter as clinically indicated.

Previous vaccination site evaluation can be done prior to administration of the current vaccination.

Patients will be followed monthly following the last vaccination visit at Week 25. Evaluations will occur once a month through Month 12 or as clinically indicated on the following. A clinical assessment (a limited, problem-oriented physical exam will be performed, including weight and vital signs); ECOG performance status; CBC, CMP and PSA; assessment of all previous sites of vaccination, immunological monitoring to include an additional 4.0 ml purple-top tube for CD4 and CD8 T cell counts; and staging tests as clinically indicated.

### Long Term Follow-Up (Starting Month 15)

Patients will be followed every three months or as clinically indicated for survival. The following assessments will be performed. The date of disease progression will be documented. A limited, problem-oriented physical exam will be performed, including weight and vital signs. The following tests will also be performed ECOG performance status, CBC, CMP and PSA, assessment of all previous sites of vaccination, immunological monitoring for CD4 and CD8 T cell counts.

Clinically significant responses to immunotherapy may take several months to develop. In studies of CG1940 and CG8711 in patients with HRPC, early PSA progression has been followed by stabilization of PSA or decreased PSA velocity in a subset of patients.

### Measurement of Effect

Response and progression can be evaluated using the international criteria proposed by the Response Evaluation Criteria in Solid Tumors (RECIST) Committee. Changes in only the largest diameter (unidimensional measurement) of the tumor lesions are used in the RECIST criteria. Lesions are either measurable or non-measurable using the criteria provided below.

Measurable disease/lesions are those that can be accurately measured in at least one dimension (longest diameter to be recorded) as >20 mm with conventional techniques (CT, MRI, x-ray) or as >10 mm with spiral CT scan. Tumor measurements are recorded in millimeters (or decimal fractions of centimeters).

Non-measurable disease: All other lesions (or sites of disease), including small lesions (longest diameter <20 mm with conventional techniques or <10 mm using spiral CT scan), are considered non-measurable disease. Bone lesions, leptomeningeal disease, ascites, pleural/pericardial effusions, lymphangitis cutis/pulmonitis, inflammatory breast disease, abdominal masses (not followed by CT or MRI), and cystic lesions are all non-measurable.

Target lesions (Table 7): All measurable lesions up to a maximum of five lesions per organ and 10 lesions in total, representative of all involved organs, should be identified as target lesions and recorded and measured at baseline. Target lesions are selected on the basis of their size (lesions with the longest diameter) and their suitability for accurate repeated measurements (either by imaging techniques or clinically). A sum of the longest diameter (LD) for all target lesions will be calculated and reported as the baseline sum LD. The baseline sum LD will be used as reference by which to characterize the objective tumor response.

Non-target lesions (Table 8): All other lesions (or sites of disease) are identified as non-target lesions and should also be recorded at baseline. Non-target lesions include measurable lesions that exceed the maximum numbers per organ or total of all involved organs as well as non-measurable lesions. Measurements of these lesions are not required but the presence or absence of each should be noted throughout follow-up.

All measurements are taken using a ruler or calipers. All baseline evaluations are performed as closely as possible to the beginning of treatment and not more than 4 weeks before the beginning of the treatment. Tumor lesions that are situated in a previously irradiated area will not be considered measurable, unless there is clear evidence of progression on physical exam or imaging studies.

The same method of assessment and the same technique should be used to characterize each identified and reported lesion at baseline and during follow-up. Imaging-based evaluation is preferred to evaluation by clinical examination when both methods have been used to assess the anti-tumor effect of a treatment.

Clinical lesions will only be considered measurable when they are superficial (e.g., skin nodules and palpable lymph nodes). In the case of skin lesions, documentation by color photography, including a ruler to estimate the size of the lesion, is recommended.

Lesions on chest x-ray are acceptable as measurable lesions when they are clearly defined and surrounded by aerated lung. However, CT can also be used.

Conventional CT and MRI are performed with cuts of 10 mm or less in slice thickness contiguously. Spiral CT should be performed using a 5 mm contiguous reconstruction algorithm. This applies to tumors of the chest, abdomen, and pelvis. Head and neck tumors and those of extremities usually require specific protocols.

When the primary endpoint of the study is objective response evaluation, ultrasound (US) is generally not used to measure tumor lesions. It is, however, a possible alternative to clinical measurements of superficial palpable lymph nodes, subcutaneous lesions, and thyroid nodules. US can be useful to confirm the complete disappearance of superficial lesions usually assessed by clinical examination.

Endoscopy and laparoscopy can be useful to confirm complete pathological response when biopsies are obtained, but will not be used for tumor measurements.

Tumor markers alone are generally not used to assess response. If markers are initially above the upper normal limit, they must normalize for a patient to be considered in complete clinical response. Specific additional criteria for standardized usage of prostate-specific antigen (PSA) and CA-125 response in support of clinical trials are being developed.

Cytology and histology can be used to differentiate between partial responses (PR) and complete responses (CR) in rare cases (e.g., residual lesions in tumor types, such as germ cell tumors, where known residual benign tumors can remain). The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.

TABLE 7
Response Criteria; evaluation of target lesions

| | |
|---|---|
| Complete Response (CR) | Disappearance of all target lesions |
| Partial Response (PR) | At least a 30% decrease in the sum of the |
| | longest diameter (LD) of target lesions, taking |
| | as reference the baseline sum LD |
| Progressive Disease (PD) | At least a 20% increase in the sum of the LD of |
| | target lesions, taking as reference the smallest |
| | sum LD recorded since the treatment started or |
| | the appearance of one or more new lesions |
| Stable Disease (SD) | Neither sufficient shrinkage to qualify for PR |
| | nor sufficient increase to qualify for PD, taking |
| | as reference the smallest sum LD since the |
| | treatment started |

TABLE 8
Evaluation of non-target lesions

| | |
|---|---|
| Complete Response | Disappearance of all non-target lesions and |
| (CR): | normalization of tumor marker level |
| Incomplete Response/ | Persistence of one or more non-target lesion(s) |
| Stable Disease (SD) | and/or maintenance of tumor marker level |
| | above the normal limits |
| Progressive Disease (PD) | Appearance of one or more new lesions and/or |
| | unequivocal progression of existing non-target |
| | lesions |

Although a clear progression of “non-target” lesions only is exceptional, in such circumstances the opinion of the treating physician should prevail, and the progression status should be confirmed at a later time.

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

In some circumstances, it may be difficult to distinguish residual disease from normal tissue. When the evaluation of complete response depends on this determination, it is recommended that the residual lesion be investigated (fine needle aspirate/biopsy) before confirming the complete response status.

### Confirmatory Measurement/Duration of Response

To be assigned a status of PR or CR (Table 9), changes in tumor measurements are confirmed by repeat assessments that should be performed between 4 and 8 weeks after the criteria for response are first met. In the case of SD, follow-up measurements must have met the SD criteria at least once after study entry at a minimum interval of eight weeks.

TABLE 9
Subject classification

| Target | Non-Target | | |
|---|---|---|---|
| Lesions | Lesions | New Lesions | Overall Response |
| CR | CR | No | CR |
| CR | Incomplete | No | PR |
| | response/SD | | |
| PR | Non-PD | No | PR |
| SD | Non-PD | No | SD |
| PD | Any | Yes or No | PD |
| Any | PD | Yes or No | PD |
| Any | Any | Yes | PD |

Note:
Patients with a global deterioration of health status requiring discontinuation of treatment without objective evidence of disease progression at that time should be classified as having “symptomatic deterioration.”

The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that recurrent disease is objectively documented.

Stable disease is measured from the start of the treatment until the criteria for progression are met, taking as reference the smallest measurements recorded since the treatment started.

### Example 10

### Determining the Immunological Effects of CD4 Modulation

This example describes methods of determine the immunological effects of CD4 modulation on the frequency, number and function of FOXP3+Treg cells as well as antigen-specific T cell responses to a variety of prostate cancer antigens relevant to GVAX vaccination.

### The Frequency and Function of FOXP3+T_{reg} Cells

It is expected that vaccination of lymphopenic patients reconstituted with CD25-depleted PBMC, treated by partial transient in vivo depletion with anti-CD4 mAb, or the combination of both (see Example 9), will lead to a reduced frequency and number of FOXP3⁺ PBMC. As shown in FIGS. 8-10, in animal models, depletion of CD25⁺ cells from the TBM spleen cells used in RLM recovered the generation of tumor-specific effector T cells, as measured by IFN-γ secretion and therapeutic in adoptive transfer experiments.

Intracellular analysis of FoxP3 expression in PBMCs can be performed as follows. Cryopreserved PBMCs from pre and post vaccine apheresis (see Example 9) are thawed and stained with surface marker-specific mAb (as shown in FIG. 11), washed, fixed and permeabilzed in perm/fix/block buffer (ebioscience, according to manufacturer's conditions), washed and stained with Phycoerythrin (PE)-conjugated anti-human FOXP3 mAb (14-5779-73 ebioscience, San Diego, Calif.), washed and analyzed in a multi-parameter flow analysis with the 9-color CyAN Instrument. A cumulative analysis of the mean FOXP3⁺/CD25⁺ frequency of CD4 T cells from one patient on this trial gave the following result: mean FOXP3+ cells pre=5.33±0.76 (CV=14.2%) and post=4.56±0.27 (CV=6%).

The function of T_{reg} can be determined by their ability to inhibit anti-CD3 stimulated proliferation. This assay can be performed on apheresis samples. CD4⁺CD25⁻ and CD4⁺CD25⁺ T cells were purified by magnetic separation with MACS (Miltenyi Biotec) following the same procedure used in Example 6. In the polyclonal T_{reg} suppressor assay, CD4⁺CD25⁻ T cells or CD8⁺T cells (5×10⁴) were cultured in media only or stimulated with immobilized anti-CD3 for 2 or 3 days. To triplicate wells, in 96-well plates, escalating doses of CD4⁺CD25⁺ cells are added. Proliferation was assessed by incorporation of [3H]thymidine (1 μCi/well), which is added for the last 16 hours of culture. Results show that functional (suppressive) T_{regs} are present in peripheral blood of vaccinated patients. Alternatives assays to monitor T_{reg} function are known in the art (e.g. FACS sorting of T_{reg} and CFSE dilution).

It is expected that patients reconstituted with CD25-depleted PBMC and/or partial and transient in vivo CD4 depletion will have fewer CD3⁺/CD4⁺/CD25⁺/FOXP3⁺ cells at all post vaccination time points, compared to Cohort B (cyclophosphamide 350 mg/m²×3, reconstitution with total PBMC and biweekly vaccination). Additionally, in vitro stimulation with anti-CD3 and anti-CD28 might be used to stimulate expression of FOXP3 in the CD25⁻ subset. If tolerance to the vaccine develops over the time course of vaccinations, an increase in detectable FOXP3 expression over the total number of CD4⁺CD25⁺ T_{reg} cells is expected.

### Antigen-Specific T Cell Responses to Prostate Cancer Antigens Relevant to GVAX

It is expected that depletion of CD25⁺ cells from the PBMC used to reconstitute the lymphopenic patient, or partial transient in vivo depletion of CD4 T cells, or the combination of both, will result in increased priming, expansion and persistence of tumor-specific T cells in the PBMC.

DCs are generated as previously described. Briefly, elutriated monocytes are cultured in X-Vivo 15 medium supplemented with 5% Human AB serum, 1000 U/ml GM-CSF and 500 U/ml IL-4 for 7 days at 37° C. Harvested DC are typically greater than 90% CD11c⁺/HLA-DR⁺/lineage negative. Immature autologous DC are transduced with lentiviral vectors encoding genes that patients vaccinated with prostate GVAX have made humoral immune responses against. The process of transducing the DC with lentiviral vectors matures the DCs and makes them excellent antigen-presenting cells.

PBMC, cryopreserved in Human albumin, X-Vivo-15 and DMSO, are thawed counted and re-suspended in X-Vivo 15 medium and plated into 24 well plates that have been coated with anti-CD3 (10 μg/ml, Ortho OKT-3). Two days later activated T cells are harvested, counted, re-suspended at 10⁵ cells/ml in X-Vivo 15 medium containing 60 Iu/ml IL-2 (Novartis) and plated into 6 well plates for 5 to 6 days culture with 5% CO2 at 37° C. Effector T cells are harvested and assayed for functional activity against autologous DC transduced with control (GFP vector) or specific prostate antigen vectors. For example, the frequency of tumor-specific IFN-γ secreting T cells (ICS), and the amount of autologous tumor-specific IFN-γ released (ELISA) can be determined. These supernatants can also be used to detect other cytokines released in response to specific tumor, the frequency of autologous tumor-specific TNF-α secreting T cells (ICS). ICS assays will counter stain with anti-CD3 and anti-CD4 or anti-CD8. Tumor-specific expression of CD107a/b can be determined and correlated with tumor-specific cytotoxicity detected in ⁵¹Cr-release assays.

### Monitoring the Patients' Response to Vaccination

The following methods can be used to identify tumor-specific T cells in a population. Table 10 provides examples of antigens known to be expressed by many prostate cancers. Using these genes/peptides/proteins as targets, it is possible to monitor a prostate specific immune response. The initial pre-vaccine IML apheresis product will be split in two parts. Half will be used to isolate PBMC and the other half elutriated (using the Gambro Elutra) to isolate monocytes. Typically, 6.0×10⁸ monocytes are cultured in GM-CSF and IL-4 to generate DC which are cryopreserved. The remaining monocytes [range of 7.8×10⁸ to 2.0×10⁹ for first 7 patients on current trial] are cryopreserved directly following isolation. Cells are cryopreserved in Human Albumin, x vivo-15 medium and DMSO. DC are pulsed with recombinant protein or are lipofected/electroporated with gene constructs. These are then used as targets. Therefore, it is possible to monitor a prostate antigen-specific response in men receiving the GVAX vaccine.

TABLE 10
Prostate tumor-associated antigens

| antigen | gene ID | kDa |
|---|---|---|
| Prostase | KLK4 | 25 |
| PSA | KLK3 | 28 |
| PAP | ACPP | 45 |
| NY-ESO-1 | CTG1B | 18 |
| LAGE-1a | CTAG2 | 21 |
| p53 | TP53 | 53 |
| Prostein | PCANAP6 | 59 |
| PSMA | FOLH1 | 100 |
| Her2/neu | ERBB2 | 137 (185) |
| Survivin | BIRC5 | 16 |
| Telomerase | TERT | 127 |

In order to identify tumor-specific T cells and maintain the in vitro viability, CD107a/b staining can be used. CD107a/b are present in the membranes of cytotoxic granules and associate with the cell surface as a result of degranulation. Although primarily associated with CD8 T cell responses, it can be used in monitoring CD4 responses. PBMCs are cultured from a patient vaccinated with GVAX (or other desired vaccine). In vitro expanded PBMCs are stimulated with a peptide (0.001-1 μg/ml) shown in Table 10 (or other appropriate peptide) and analyzed for tetramer and CD107a/b expression 6 hours later using anti-CD107a/b antibody. As the concentration of peptide is increased, a high percentage of the tetramer⁺ cells are triggered by the peptide. In this case, the “response” is expression of CD107a/b.

Another method that can be used is to detect T cells that respond to whole tumor cells. PBMCs from a vaccinated patient is activated with anti-CD3 and expanded with 60 IU/ml IL-2 for an additional 5 days. This generates effector T cells from PBMC (or TVDLN). T cells generated in this way can be assayed for tumor-specific activity by stimulation with specific tumor or peptide. Stimulated T cells can be assayed for IFN-γ secretion (for example by ELISA) and stained with anti-CD107a/b. Therefore, CD107a/b staining permits identification of the frequency of tumor-reactive T cells. Since CD107a/b staining does not affect the viability, this approach can be used to isolate/sort tumor-reactive T cells for functional or microarray analysis.

### Example 11

### Treatment of a Tumor or Pathogen In Vivo

This example describes methods that can be used to treat a tumor, by administration of an agent that depletes CD4+ T cells, subsequent to administration of a first dose of a cancer vaccine. One skilled in the art will appreciate that similar methods can be used to treat any type of tumor, by administration of a cancer vaccine that includes tumor antigens specific for that tumor. Similarly, one skilled in the art will appreciate that similar methods can be used to treat any type of pathogen, by administration of a vaccine that includes antigens specific for the pathogen of interest. In such examples, the vaccine may be prophylactic.

Generally, the method includes vaccinating subjects having a tumor (or a subject who has had a tumor removed) with a first dose of a therapeutically effective amount of a cancer vaccine that includes TAAs expressed by the tumor in the subject (for example on day 7). Although it is also appreciated that the tumor in the patient may serve as the patient's own (in situ) vaccine if the T_{reg} cell numbers are reduced/eliminated. In this instance, only anti-CD4 needs to be administered alone or together with cytokines (e.g., GM-CSF, IL-2) or the co-stimulatory agents (e.g. anti-CD134, anti-CTLA-4, anti-4-1BB). For example, if the subject has or had colon cancer, the cancer vaccine includes colon cancer TAAs (such as the ALVAC CEA B.71 vaccine for colorectal cancer). The cancer vaccine selected will depend on the subject's tumor. In one example where the cancer vaccine includes cells, at least 1×10⁶ cells are administered, such as at least 1×10⁸ cells. Subsequently, the subject is administered a therapeutically effective amount of an agent that depletes CD4+ T cells in the subject (for example by at least 30%-80%), such as an anti-CD4 humanized monoclonal antibody. The amount of antibody added is one that achieves a reduction of CD4+ T cells of at least 30%, such as 30-80%. In particular examples, the treatment does not deplete all CD4+ T cells.

In some examples, the subject is subjected to peripheral blood mononuclear cell (PBMC) harvest (for example on days −5 and −10), followed by the induction of non-myloablative lymphodepletion (such as days 1-3), and reconstitution with an autologous PBMC infusion (such as day 6). A particular example of non-myloablative lymphodepletion is administration of 100-500 mg/m² IV of cyclophosphamide (such as 200-400 mg/m²) on three consecutive days, administration of 1-30 mg/m² IV of fludarabine (such as 10-25 mg/m²) on three consecutive days, or combinations thereof. In some examples, the autologous PBMC infusion has been depleted of CD25, CD81, or both types of cells.

For example, the subject has leukapheresis. The resulting WBC are frozen and can be subjected to iT_{reg} depletion (for example CD25 T cell or CD81 T cell depletion, or both). The PBMCs or depleted PBMCs are later returned to the subject. After the leukapheresis, subjects are treated with therapeutically effective amounts of cyclophosphamide chemotherapy (for example in combination with fludarabine) infusion into a vein. On the sixth day the subject's frozen white blood cells are infused into the subject intravenously. The following day, the vaccine containing TAAs is administered. Subsequently, the subject is administered a CD4 T cell depleting agent, for example on weeks 3, 7 and 11. In addition, the subject is administered booster doses every two weeks, for example for 6 months.

X-rays and scans can be performed at least every 13 weeks to monitor the tumors.

### Baseline Leukapheresis

Subjects undergoing lymphodepletion can begin leukapheresis within two weeks of the start of lymphodepeltion to obtain PBMC for reconstitution and for immune monitoring. At least 1×10¹⁰ PBMC are obtained. Approximately 10 liters of blood are processed over 3-6 hours. The median yield of PBMC from a 2.5 hour leukapheresis that processed a median of 7.7 liters was 8.35×10⁹ PBMC, of which 25% are monocytes.

Calcium gluconate (10 ml) in 100 ml normal saline (NS) is infused at 0.5 ml/min or 30 ml/hr during the procedure. Subjects whose peripheral access is inadequate can have a temporary hemodialysis catheter placed under direct ultrasound guidance. The catheter will be removed after the leukapheresis procedure.

After pheresis, the product is separated into lymphocyte and monocyte fraction by elutriation with adapted MNC protocol (Rouard et al., Transfusion 43:481-7, 2003). Lymphocytes will be processed and frozen for later autologous re-infusion according to ARC standard procedure for Hematopoietic Progenitor Cell processing, storage and re-infusion. At the time of reinfusion, a sample of the product will be used for CBC with differential to determine the number of lymphocytes reinfused into each subject.

If mild symptomatic hypocalcemia occurs during leukapheresis (tingling of lips/face, numbness in extremities, muscle cramps), oral Turns will be given as needed. For subjects who become neutropenic and febrile, empiric antibiotic treatment with ceftazadime or imipenem can be given. Packed red blood cell transfusions are given if subjects have symptomatic anemia or if their Hgb less than 8 g/dl. Platelet transfusions can be given if the platelet count falls below 10,000 μL or at higher levels if evidence of bleeding is present.

A baseline CBC can be performed within 3 days of the procedure. Ideally, subjects who will receive the vaccine will have a WBC≧3,000; platelet count≧100,000; Hgb≧8 g/dl; and Hc greater than 24%

### Chemotherapy

Prior to vaccination with TAAs and infusion of PBMCs, chemotherapy may be administered according to guidelines based on early adoptive immunotherapy trials (Dudley et al. Science 298:8504, 2002; Rosenberg and Dudley, Proc. Natl. Acad. Sci. USA 101:14639-45, 2004). However, subjects need not receive chemotherapy.

Cyclophosphamide 100-500 mg/m²/day IV (such as 350 mg/m²/day IV) over one hour each day for 3 consecutive days (days 1-3), 1-30 mg/m² IV of fludarabine (such as 20 mg/m²), or combinations thereof, is administered on three consecutive days. Hydration and antiemetics (excluding dexamethasone) can be used at the treating physician's discretion, for example to prevent nausea.

Cyclophosphamide (2-[bis(2-chloroethyl)amino]tetrahydro-2H-1,3,2 oxazaphosphorine 2-oxide monohydrate) is a synthetic antineoplastic drug with the molecular formula C₇H₁₅Cl₂N₂O₂P.H₂O and a molecular weight of 279.1. Lyophilized CYTOXAN® (cyclophosphamide for injection, USP) contains 75 mg mannitol per 100 mg cyclophosphamide (anhydrous) and can be reconstituted with sterile water or normal saline. For example, CYTOXAN® will be diluted in about 150 cc of normal saline and infused IV over 30-60 minutes. An added dose of IV fluids may help prevent bladder toxicity. Although the reconstituted cyclophosphamide is stable for six days under refrigeration, it contains no preservatives and therefore ideally is used within 6 hours.

CYTOXAN® Tablets (cyclophosphamide tablets, USP) are for oral use and contain 25 mg or 50 mg cyclophosphamide (anhydrous). Cyclophosphamide is well absorbed after oral administration with a bioavailability greater than 75%. The unchanged drug has an elimination half-life of 3 to 12 hours.

### Vaccination

Beginning on day 4 (one day after the last dose of chemotherapy, if chemotherapy is administered, day 1 is no chemotherapy is administered), subjects are immunized intradermally with the desired vaccine, at the desired dose. Cancer and pathogen vaccines are known, and include the use of DRibbles (either direct administration or administration of APC or DC cells loaded with DRibbles). Vaccinations can be rotated among all extremities. The abdomen and flank can also be used. Subjects can be observed for fifteen minutes after each vaccination.

The vaccinations can be repeated every two weeks for six months unless conditions for discontinuation are met. However, one skilled in the art will appreciate that other regimens can be used, such as vaccinations every month. Subjects will not be retreated if any acute systemic toxicity greater than grade 2 attributable to the vaccine administration occurs. Subjects are allowed to continue receiving vaccine for grade 2 toxicities commonly associated with the vaccine including skin rash, fever, malaise, adenopathy and local reactions. If severe local toxicity such as ulceration or sterile abscess occurs, the dose of the vaccine will be decreased in subsequent vaccines to 50% of initial dose. If the toxicity recurs at the lower dose, then the vaccines will be discontinued. Unexplained visual changes detected clinically will result in discontinuation of the vaccine because of the possibility that the vaccine induced a response to pigmented cells within the retina.

If manifestations of auto-immune disease occur (such as inflammatory arthritis, vasculitis, pericarditis, glomerulonephritis, erythema nodusum), then appropriate medical management will be offered including non-steroidal anti-inflammatory agents, steroids, or other immunosuppressive medications as dictated by the clinical situation and vaccination discontinued.

### Depletion of CD4+ T Cells

Following the priming vaccination, the subject is administered a therapeutically effective amount of an agent that depletes CD4+ T cells in the subject (for example by at least 30%-80%). One particular example of such an agent is an anti-CD4 humanized monoclonal antibody. The amount of antibody added is one that achieves a reduction of CD4+ T cells of at least 20%, such as at least 50%, at least 75%, for example 20-80% or 20-50%. In particular examples, the treatment does not deplete all CD4+ T cells. The concept is to “tip the balance” of CD4 T cell helper activity and CD4 T cell regulatory activity away from suppressive function and in favor of activity that “helps” support a therapeutic anti-cancer immune response.

In a particular example, the anti-CD4 antibody is HuMax-CD4 (Genmab, Denmark), and it administered s.c. at a dose of 20-200 mg, such as 50-100 mg, 40-90 mg, or 80 mg (for example on weeks. 3, 7 and 11, such as on the days booster vaccinations are administered). In another example, the anti-CD4 antibody is zanolimumab, and is administered at a dose of about 0.1-5 mg/kg i.v. on weeks 3, 7 and 11, just prior to the vaccination with the booster (for example a dose of 0.1-2 mg/kg, 0.5-2 mg/kg, 0.5-1.5 mg/kg, such as 1 mg/kg).

### Autologous Peripheral Blood Mononuclear Cell Infusion

In some examples, on day 6, subjects are also infused with their previously frozen autologous PBMC. In particular examples, such PBMCs are treated ex vivo to deplete iT_{regs}, for example by depleting CD25+, CD81+, Areg⁺, Ptgr3⁺, or CD134⁺ cells (or combinations thereof). In particular examples, CD25+, CD81+, Areg⁺, Ptgr3⁺, or CD134⁺ cells (or combinations thereof) are depleted by at least 20%, at least 30%, at least 50%, at least 80%, or at least 95%, for example 20-95%. For example, the methods described in Examples 6-8 can be used.

Premedication can include acetaminophen (650 mg) and diphenhydramine (50 mg) by mouth 30 minutes before the PBMC infusion. The minimum number of PBMCs that will be infused can be 10⁶-10¹⁰, such as 4×10⁹. In one example, the maximum number of PBMCs infused is 10¹¹. The PBMC will be infused IV push over five minutes for each 50 cc syringe. The cell infusions will be given through a large bore IV line suitable for a blood transfusion without a filter.

The subject is hydrated for 4 hours before and for 4-6 hours following the PBMC infusion to help protect against renal failure. Hydration will be adjusted to insure urine output of at least 100 ml/hr. Hydration will be achieved by infusing D₅W½NS+20 mEq KCl+50 mEq NaHCO₃ per liter at a rate of 150 ml/hr.

Vital signs will be obtained at baseline, after 20 cc have infused and at the end of the infusion. Thereafter, vital signs will be obtained every thirty minutes for two hours and then every hour for four hours. From the day after reinfusion until neutrophils recover to at least 1,000 μL and lymphocytes to at least 500/μL, subjects are monitored for infection.

### Duration of Therapy

In the absence of treatment delays due to adverse events, treatment can continue until one or more of the following occurs: disease progression (subjects without progression can continue treatment for at least one year); intercurrent illness that prevents further administration of treatment; unacceptable adverse event(s); subject decides to terminate treatment; or changes in the subject's condition render the subject unacceptable for further treatment. In particular examples, the treatments are repeated, for example every 2 weeks, for example for up to a few years (such as up to 5 years).

Subjects will receive no additional vaccinations if they experience any of the following toxicities: grade 3 allergy/immunology, grade 3 hemolysis, grade 3 cardiac, grade 3 coagulation, grade 3 endocrine, grade 3 gastrointestinal, grade 4 infection, grade 3 metabolic, grade 3 neurology, grade 3 ocular, grade 3 pulmonary, and grade 3 renal.

### Second Collection of PBMCs

If desired, subjects can undergo a second collection of mononuclear cells for analysis of immune function, such as approximately 2 weeks after the fifth vaccine. The product need not be processed for re-infusion into the subject. Approximately two weeks following the vaccination, leukapheresis for collection of PBMC can be performed over 2-3 hours. PBMCs are collected at 1 ml/min, at <3% colorgram, and over a minimum of 2 hours. The procedure does not require intravenous hydration and is generally well tolerated.

### Clinical Evaluations

A complete medical history, including previous cancer history and therapy, is obtained. A complete physical exam is performed, including height, weight and vital signs.

Tumors can be assessed as follows. A radiographic staging assessment of known sites of metastatic disease is performed within 4 weeks of day 1 chemotherapy. In addition, and MRI of the head with and without contrast can be performed.

Prior to each leukapheresis procedure, CBC parameters are evaluated.

Approximately two weeks following the fifth vaccination, subjects can undergo re-staging imaging studies to evaluate anti-tumor response. Staging is repeated after every two additional vaccines (every two months). Subjects with stable disease or better can continue vaccination for one year (up to a total of 16 vaccines).

After all vaccinations are completed, subjects can be followed every 3 months for long-term toxicity and survival for the duration of their life.

Methods for Analysis of Tumors

Subjects can be reevaluated for response after the first five vaccinations, and then after every two vaccinations (every 2 months). In addition to a baseline scan, confirmatory scans can be done 4-8 weeks following initial documentation of objective response.

Response and progression can be evaluated using the international criteria proposed by the Response Evaluation Criteria in Solid Tumors (RECIST) Committee (JNCI 92(3):205-16, 2000). Changes in only the largest diameter (unidimensional measurement) of the tumor lesions are used in the RECIST criteria. Lesions are either measurable or non-measurable using the criteria provided herein. The term “evaluable” in reference to measurability will not be used.

### Evaluation of Lesions Response to Vaccine

For target lesions, a complete response (CR) is the disappearance of all target lesions. A partial response (PR) is at least a 30% decrease in the sum of the longest diameter (LD) of target lesions, taking as reference the baseline sum LD. Progressive disease (PD) is an observation of at least a 20% increase in the sum of the LD of target lesions, taking as reference the smallest sum LD recorded since the treatment started or the appearance of one or more new lesions. Stable disease (SD) is the observation of neither sufficient shrinkage to qualify for PR nor sufficient increase to qualify for PD, taking as reference the smallest sum LD since the treatment started.

For non-target lesions, a complete response (CR) is the disappearance of all non-target lesions and normalization of tumor marker level. An incomplete response can be the observation of Stable Disease (SD), the persistence of one or more non-target lesion(s) and/or maintenance of tumor marker level above the normal limits. Progressive Disease (PD) is the appearance of one or more new lesions and/or unequivocal progression of existing non-target lesions.

To be assigned a status of PR or CR, changes in tumor measurements can be confirmed by repeat assessments performed between 4 and 8 weeks after the criteria for response are first met. In the case of SD, follow-up measurements ideally satisfy the SD criteria at least once after study entry at a minimum interval of six to eight weeks.

The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that recurrent disease is objectively documented.

Stable disease is measured from the start of the treatment until the criteria for progression are met, taking as reference the smallest measurements recorded since the treatment started.

All subjects will be assessed for response to treatment. Each subject is assigned one of the following categories: 1) complete response, 2) partial response, 3) stable disease, 4) progressive disease, 5) early death from malignant disease, 6) early death from toxicity, 7) early death because of other cause, or 9) unknown (not assessable, insufficient data). Subjects in response categories 4-9 are considered as failing to respond to treatment (disease progression).

### Endpoints (Immune Parameters, Toxicity Parameters, Tumor Responses)

Toxicity parameters will be measured primarily by counts of granulocytes and lymphocytes (number of cells per microliter). The amount of time to return to >200 lymphocytes/μL and 1000 neutrophils/μL is determined.

Several assays are known in the art that can be used to characterize T lymphocyte responses. Data from these assays are typically displayed as bivariate scatter plots on logarithmic scales, often referred to in the literature as “two-parameter histograms.” Vertical and horizontal reference lines (calibrated statistical “cursors”) divide the scatter plots into four quadrants, positive/positive events being displayed in the upper right quadrant. One primary endpoint, or criterion measure, for the immune parameters, is a percentage value (or frequency) represented by the number of CD8⁺T cells to the total number of gated CD8⁺ T lymphocytes (expressed as a percentage) that make intracellular IFN-γ. Analyses can include pairwise comparisons of intra-patient (within-subject) scores (such as pre-vaccine vs. post-vaccine frequencies). These will be continuous random variables, typically small numbers, varying from 0.05% to 5.0%.

Anti-tumor immune responses are measured before and after treatment (such as 60 days post initial vaccination). T cells from leukophoresis products before and after treatment are isolated with MACS bead by negative selection. T cells are stimulated with DRibble/DC used for vaccine, or autologous tumor cells if available in presence of a Golgi blocker that allows accumulation of cytokines inside cells.

IFN-γ production by T cells can be measured by intracellular staining techniques after cell surface staining with CD4 and CD8 antibodies. For example, T cells stimulated with a specific and non-specific tumor cell can be stained with labeled antibodies for CD4, CD8, and IFN-γ. Using flow cytometry, the signals are detected to determine the percentage of CD4 and CD8 T cells produce IFN-γ with or without stimulation are determined. The tumor-specific response is when CD8 and CD4 cells produce IFN-γ in the presence of the tumor cells, but not the non-specific tumor cells.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A method of stimulating an immune response against a target antigen, comprising:
reducing a number of CD4+ T cells in a subject subsequent to the subject receiving a first dose of a therapeutically effective amount of an immunogenic composition comprising the target antigen, wherein the subject has a tumor comprising the target antigen; and
administering a therapeutically effective amount of a second dose of the immunogenic composition to the subject, thereby stimulating an immune response against the target antigen.

2. The method of claim 1, wherein reducing a number of CD4+ T cells comprises:
administering a therapeutically effective amount of an agent that significantly reduces the number of CD4+ T cells in the subject under conditions sufficient to reduce the number of CD4+ T cells in the subject.

3. The method of claim 2, wherein the agent that significantly depletes CD4+ T cells comprises a therapeutically effective amount of an anti-CD4 antibody.

4. The method of claim 1, wherein the number of CD4+ T cells in the subject is reduced by at least 30%.

5. The method of claim 1, wherein administering the agent that reduces the number of CD4+ T cells and administering the second dose of the immunogenic composition occurs simultaneously.

6. The method of claim 1, wherein reducing the CD4+ T cells in a subject occurs at least 10 days subsequent to the subject receiving the first dose of the immunogenic composition.

7. The method of claim 1, further comprising:
administering to the subject autologous peripheral blood mononuclear cells (PBMCs) prior to or at essentially the same time when the subject received the first dose of the immunogenic composition.

8. The method of claim 7, wherein the PBMCs are substantially depleted of CD25⁺ cells, CD81⁺ cells, CD134⁺ cells, Areg⁺ cells, Ptgr3⁺ cells, or combinations thereof.

9. The method of claim 7, further comprising:
lymphodepleting the subject prior to administering the first dose of the immunogenic composition and the PBMCs, and following apheresis of the subject.

10. The method of claim 9, wherein lymphodepleting the subject comprises:
administering to the subject a therapeutically effective amount of an lymphodepletion agent, under conditions sufficient to significantly reduce the number of lymphocytes in the subject.

11. The method of claim 10, wherein the lymphodepletion agent comprises one or more anti-neoplastic chemotherapeutic agents, radiation therapy, or combinations thereof.

12. The method of claim 11, wherein the one or more anti-neoplastic chemotherapeutic agent comprises therapeutically effective amounts of cyclophosphamide, Fludarabine, o radiation therapy, r combinations thereof.

13. The method of claim 10, wherein administering the therapeutically effective amount of the lymphodepletion agent comprises administering multiple doses of the lymphodepletion agent or radiation therapy to the subject.

14. The method of claim 1, wherein the subject is a mammal.

15. The method of claim 14, wherein the mammal is a human.

16. The method of claim 14, wherein the mammal is a veterinary subject.

17. The method of claim 1, wherein the tumor is a breast cancer, a melanoma, a lung cancer, a renal cell carcinoma, a prostate cancer, an ovarian cancer, a cervical cancer, a colon cancer, a liver cancer, or combinations thereof.

18. The method of claim 1, wherein the method treats the tumor.

19. A method of stimulating an immune response against a tumor cell in a subject, comprising:
administering to the subject a therapeutically effective first dose of an immunogenic composition comprising one or more tumor antigens associated with the tumor cell;
administering a therapeutically effective dose of anti-CD4 to the subject, thereby reducing CD4+ cells in the subject by at least 30%; and
administering to the subject a therapeutically effective second dose of the immunogenic composition, thereby stimulating an immune response against the tumor cell in the subject.

20. A method of stimulating an immune response against a tumor cell in a subject, comprising:
isolating PBMCs from the subject;
lymphodepleting the subject;
administering a therapeutically effective dose of the PBMCs to the subject;
administering to the subject a therapeutically effective first dose of an immunogenic composition comprising one or more tumor antigens associated with the tumor cell;
reducing CD4+ T cells in the subject by at least 30%; and
administering to the subject a therapeutically effective second dose of the immunogenic composition, thereby stimulating an immune response against the tumor cell in the subject.

21. The method of claim 20, wherein the PBMCs administered to the subject are significantly depleted of CD25⁺ cells, CD81⁺ cells, CD134⁺ cells, Areg⁺ cells, Ptgr3⁺ cells, or combinations thereof.

22. The method of claim 20, wherein administering the second dose of the immunogenic composition to the subject comprises administration of at least three doses of the immunogenic composition over a period of at least 180 days.

23. The method of claim 20, further comprising:
reducing the number of CD4+ T cells in a subject by at least 30%; and
administering to the subject a third dose of the immunogenic composition, thereby stimulating an immune response against the tumor cell in the subject.

24. A kit comprising:
an anti-CD4 antibody; and
an anti-CD25 antibody, an anti-CD81 antibody, an anti-CD134 antibody, an anti-Areg antibody, an anti-Ptgr3 antibody, or combinations thereof.

25. The kit of claim 24, further comprising an immunogenic composition.

26. The kit of claim 25, wherein the immunogenic composition comprises a cancer vaccine.

27. The kit of claim 24, further comprising a chemotherapeutic agent.


SEQUENCE LISTING (1 sequence)

SEQ ID NO: 1 - RNA, 20 nt, Human immunodeficiency virus
gcccgucuguugugugacuc